(19) United States
(12) Patent Application Publication
(10) Pub. No.: US 2026/0256601 A1
(43) Pub. Date: Sep. 3, 2026

(54) PROSTHETIC HAND

(71) Applicant: METACARPAL LTD, Edinburgh (GB)

(72) Inventors: Fergal Joseph Donald Mackie, Edinburgh (GB); Christopher James Nisbet, Edinburgh (GB)

(73) Assignee: METACARPAL LTD, Edinburgh (GB)

(21) Appl. No.: 19/100,113

(22) PCT Filed: Nov. 15, 2024
(86) PCT No.: PCT/EP2024/082525

§ 371 Date: Jan. 30, 2025

(30) Foreign Application Priority Data
Nov. 15, 2023 (GB) 2317532.6

(51) Int. Cl. A61F 2/58; A61F 2/50; A61F 2/68

(52) U.S. Cl. CPC A61F 2/586; A61F 2/68; A61F 2002/5093

(57) ABSTRACT

A prosthetic hand comprises a hand body, a plurality of fingers and a mechanism for actuating movement of the plurality of fingers relative to the hand body. The mechanism comprises: a movable primary drive element comprising one or more primary drive pulleys; and a plurality of movable secondary drive elements each comprising a corresponding secondary drive pulley. Each of the plurality of secondary drive elements is coupled to the primary drive element by a coupling cable supported by the primary and secondary drive pulleys, such that movement of the primary drive element in a driving direction away from the secondary drive elements exerts a force on the secondary drive elements urging movement of the secondary drive elements in the driving direction. Each finger of the plurality of fingers is coupled to a corresponding secondary drive element of the plurality of secondary drive elements such that movement of a secondary drive element in the driving direction drives movement of the corresponding finger in a first direction relative to the hand body. The primary drive element and the secondary drive elements are mounted on one or more guides which constrain the direction of movement of the said primary drive element and the secondary drive elements.

### FIELD

The present disclosure concerns prosthetic hands, methods of operating prosthetic hands, kits of parts for assembling prosthetic hands, and methods of assembling and/or disassembling prosthetic hands.

### BACKGROUND

A prosthesis is an artificial device that replaces a body part which may be missing, for example, due to trauma, disease, amputation or a congenital disorder.

A person who is missing a hand may use a prosthetic hand or other terminal device (such as a hook) which is typically attached to a socket worn on the body (for example, supported by a harness).

Prosthetic hands or other terminal devices can be body-powered (where movement of device components is driven by movements of the user's body) or electrically powered (also known as “myoelectric”, where movement of device components is driven by electric motors). Such devices may be cable-operated, where movement of device components is driven by applying tension to operating cables. Both body-powered and electrically powered devices may be cable-operated.

Human fingers are typically movable in two different modes by action of the muscles of the hand: flexion and extension; and abduction and adduction. Human fingers are also typically individually movable, i.e. independently or at least partially independently of one another.

Body-powered and electrically powered prosthetic hands have been developed which comprise fingers moveable in at least the flexion/extension mode. Depending on the prosthetic hand design, some or all of the fingers may be movable individually or together in unison. However, movement mechanisms for such prosthetic hands can be complex, making replacement or repair of prosthetic hands or components thereof difficult and/or expensive. In addition, it can be difficult to balance individualised finger control with the need to apply sufficient force through the fingers to carry out common manual tasks. Improved prosthetic hands would therefore be desirable.

### SUMMARY OF INVENTION

According to a first aspect, there is provided a prosthetic hand comprising a hand body, a plurality of fingers and a mechanism for actuating movement of the plurality of fingers relative to the hand body. The mechanism comprises: a movable primary drive element comprising one or more primary drive pulleys; and a plurality of movable secondary drive elements each comprising a corresponding secondary drive pulley. Each of the plurality of secondary drive elements is coupled to the primary drive element by a coupling cable supported by the primary and secondary drive pulleys, such that movement of the primary drive element in a driving direction away from the secondary drive elements exerts a force on the secondary drive elements urging movement of the secondary drive elements in the driving direction. Each of the plurality of secondary drive elements is coupled to a corresponding finger of the plurality of fingers such that movement of a secondary drive element in the driving direction drives movement of the corresponding finger in a first direction relative to the hand body. The primary drive element and the secondary drive elements are mounted on one or more guides which constrain the direction of movement of the said primary drive element and the secondary drive elements.

In use, application of a force to the primary drive element, which causes movement of the primary drive element in the driving direction, exerts a corresponding force on the secondary drive elements which urges movement of the secondary drive elements in the driving direction. When one or more of the fingers coupled to a secondary drive element is able to be moved in the first direction relative to the hand body (for example, because movement of the said one or more fingers is not blocked by an object), exertion of the force on the secondary drive elements causes movement of the one or more secondary drive elements corresponding to the said one or more fingers in the driving direction, which in turn drives movement of the one or more fingers in the first direction relative to the hand body. However, when one or more of the fingers coupled to a secondary drive element is unable to be moved in the first direction relative to the hand body (for example, because movement of the said one or more fingers is blocked by an object), the one or more secondary drive elements corresponding to the said one or more fingers do not move in the driving direction despite the exertion of the force thereon. The mechanism for moving the fingers may therefore be considered a differential mechanism.

The use of pulleys to drive movement of the fingers also enables amplification (by the principle of mechanical advantage) of the force applied to cause the movement of the primary drive element. The mechanism for actuating movement of the plurality of fingers relative to the hand body can therefore also be considered a force amplification system and the primary drive element can be considered a primary force amplification element.

Because the primary drive element and the secondary drive elements are mounted on one or more guides which constrain the direction of movement of the said primary drive element and the secondary drive elements, movement of the primary drive element and the secondary drive elements may be smoother, more stable and/or more consistent, and the likelihood of jamming of the mechanism and/or tangling of the coupling cable or any other cabling components may be reduced. This may in turn lead to better (e.g. smoother and more reliable) control of finger movement and a reduced likelihood of malfunction.

It will be appreciated that terms such as “urge movement” and “urging movement” indicate that a force applied to a body will act so as to cause movement of that body in the direction indicated if the body is free to move. However, when a force is exerted on a body so as to urge movement of the body in a particular direction, the body may not actually move in that direction if movement is blocked (e.g. by the presence of another object)—the body will only move under the force when any impediment to movement is removed or overcome.

The fingers are movable relative to the hand body.

It may be that each finger is movable between at least two different positions corresponding to at least two different finger flexion configurations. It will be appreciated that the term “finger flexion configuration” refers to a configuration the finger would take in the flexion-extension mode of movement (analogous to the typical flexion-extension mode of movement of the fingers of a human hand). Thus, it will be appreciated that a finger flexion configuration is not necessarily a flexed configuration but may be an extended configuration. Thus, the term “finger flexion configuration” could be replaced by the equivalent terms “flexion-extension configuration” or “extension configuration”.

Movement of a finger in the first direction relative to the hand body may therefore change the finger flexion configuration of the finger. For example, movement of the finger in the first direction relative to the hand body may comprise or correspond to flexing or extending the finger. Flexing the finger may comprise moving a distal tip of the finger closer to the hand body (e.g. corresponding to a movement of a human finger in which the finger curls towards the palm). Extending the finger may comprise moving the distal tip of the finger away from the hand body (e.g. corresponding to a movement of a human finger in which the finger uncurls away from the palm towards an extended, i.e. pointing, position).

Movement of each finger may comprise hinging or pivoting of the finger or a portion thereof about a hinge joint, for example, where the finger connects to the hand body (e.g. corresponding to a knuckle of a human hand). Movement of each finger may comprise bending or unbending of the finger or a portion thereof. Each finger may be articulated and movement of each finger may comprise articulated movement of said finger or a portion thereof.

The plurality of fingers may comprise at least three fingers or at least four fingers. For example, the plurality of fingers may be three fingers or may be four fingers.

One or more (e.g. some, for example, all) of the plurality of fingers may correspond to the fingers of a human hand.

The plurality of fingers may comprise an outermost finger which corresponds to a little finger (otherwise known as a pinkie finger) of a human hand. It will be appreciated that the little finger of the human hand is the finger of the hand positioned closest to the posterior of the human body when a person is standing upright, the arm is hanging in a neutral position at the person's side and the hand is turned such that the palm faces the body (e.g. the closest leg).

The prosthetic hand may comprise a thumb, i.e. a finger which corresponds to the thumb of a human hand. It will be appreciated that the thumb of the human hand is the finger of the hand positioned closest to the anterior of the human body when a person is standing upright, the arm is hanging in a neutral position at the person's side and the hand is turned such that the palm faces the body (e.g. the closest leg). The thumb may be provided in addition to the plurality of fingers movable by the mechanism for actuating movement. The thumb may be removably mounted or mountable on the hand body. The thumb may be fixed in position or the thumb may be movable. The thumb may be opposable or opposed relative to the plurality of fingers.

The prosthetic hand may further comprise a thumb mechanism for actuating movement of the thumb. For example, the thumb may be coupled to the primary drive element via a thumb cable supported by a thumb pulley. It may be that movement of the primary drive element in the driving direction exerts a force on the thumb driving movement of the thumb in a first direction. It may be that movement of the primary drive element in the driving direction causes coordinated movement of the thumb and the index finger to generate a pinching movement.

The prosthetic hand (e.g. the plurality of fingers) may comprise a finger which corresponds to an index finger of a human hand. The index finger may be the finger closest to the thumb.

In some embodiments, the prosthetic hand comprises four fingers movable by the mechanism for actuating movement of the plurality of fingers and a thumb (optionally movable by the thumb mechanism). The four fingers may consist of fingers corresponding to the pinkie finger, ring finger, middle finger and index finger of the human hand.

In some embodiments, each finger of the plurality of fingers is coupled to a corresponding secondary drive element of the plurality of secondary drive elements such that movement of a secondary drive element in the driving direction drives movement of the corresponding finger in the first direction relative to the hand body.

Alternatively, it may be that not all fingers of the plurality of fingers are coupled to a corresponding secondary drive element. For example, in some embodiments, the plurality of fingers (i.e. the plurality of fingers which are coupled to the secondary drive elements) is a plurality of secondary fingers and the prosthetic hand further comprises a primary finger coupled directly to the primary drive element (i.e. not via a secondary drive element) such that movement of the primary drive element in the driving direction (i.e. directly) drives movement of the primary finger in the first direction relative to the hand body. Since the primary finger is coupled directly to the primary drive element, it may be that movement of the primary drive element in the driving direction is not possible (i.e. movement of the primary drive element in the driving direction is blocked) when movement of the primary finger in the first direction is not possible (i.e. movement of the primary finger in the first direction is blocked). The primary finger may correspond to the index finger of a human hand. The prosthetic hand may comprise three secondary fingers and one primary finger, wherein the three secondary fingers consist of fingers corresponding to the pinkie finger, ring finger and middle finger of the human hand and the primary finger corresponds to the index finger of a human hand.

Each of the plurality of fingers which is coupled to a corresponding secondary drive element may be coupled to the corresponding secondary drive element by a corresponding coupling mechanism. In embodiments in which a primary finger is coupled directly to a primary drive element, the primary finger may be coupled to the primary drive element by a corresponding coupling mechanism.

Each coupling mechanism may comprise one or more corresponding finger coupling cables.

In some embodiments, the coupling mechanisms are configured such that two or more (e.g. all) of the fingers are moved in the first direction relative to the hand body at the same rate as one another when the corresponding two or more (e.g. all) secondary drive elements (and optionally the primary drive element, in embodiments in which a primary finger is coupled directly to the primary drive element) are moved in the driving direction at the same rate as one another.

Alternatively, however, it may be that the coupling mechanisms are configured such that that two or more (e.g. all) of the plurality of fingers are moved in the first direction relative to the hand body at different rates from one another when the corresponding two or more (e.g. all) secondary drive elements (and optionally the primary drive element, in embodiments in which a primary finger is coupled directly to the primary drive element) are moved in the driving direction at the same rate as one another. For example, it may be that two or more (e.g. all) of the plurality of fingers are moved in the first direction by different amounts (e.g. through different distances or by different angular amounts, dependent on the type of movement) when the corresponding two or more secondary drive elements (and optionally the primary drive element, in embodiments in which a primary finger is coupled directly to the primary drive element) are moved in the driving direction by the same amount (e.g. when the corresponding two or more secondary drive elements (and optionally the primary drive element) are displaced through the same distance in the driving direction).

It may be that the coupling mechanism for each finger comprises a corresponding drive wheel for driving movement of the said finger in the first direction. Each drive wheel may be coupled to the corresponding secondary drive element (or optionally the primary drive element, in embodiments in which a primary finger is coupled directly to the primary drive element) by a corresponding drive cable.

In some embodiments, two or more (e.g. all) of the drive wheels are configured such that the two or more (e.g. all) of the corresponding two or more fingers are moved in the first direction relative to the hand body at the same rate as one another when the corresponding two or more (e.g. all) secondary drive elements (and optionally the primary drive element, in embodiments in which a primary finger is coupled directly to the primary drive element) are moved in the driving direction at the same rate as one another.

In some embodiments, two or more (e.g. all) of the drive wheels have the same drive radii such that the two or more (e.g. all) of the corresponding two or more fingers are moved in the first direction relative to the hand body at the same rate as one another when the corresponding two or more (e.g. all) secondary drive elements (and optionally the primary drive element, in embodiments in which a primary finger is coupled directly to the primary drive element) are moved in the driving direction at the same rate as one another.

Alternatively, however, it may be that two or more (e.g. all) of the drive wheels are configured such that the corresponding two or more (e.g. all) fingers are moved in the first direction relative to the hand body at different rates from one another when the corresponding two or more (e.g. all) secondary drive elements (and optionally the primary drive element, in embodiments in which a primary finger is coupled directly to the primary drive element) are moved in the driving direction at the same rate as one another. For example, it may be that two or more (e.g. all) of the plurality of fingers are moved in the first direction by different amounts (e.g. through different distances or by different angular amounts) when the corresponding two or more (e.g. all) secondary drive elements (and optionally the primary drive element, in embodiments in which a primary finger is coupled directly to the primary drive element) are moved in the driving direction by the same amount (e.g. when the corresponding two or more secondary drive elements (and optionally the primary drive element) are displaced through the same distance in the driving direction) due to the difference in configuration.

For example, it may be that two or more (e.g. all) of the drive wheels have different drive radii such that the corresponding two or more (e.g. all) fingers are moved in the first direction relative to the hand body at different rates from one another when the corresponding two or more (e.g. all) secondary drive elements (and optionally the primary drive element, in embodiments in which a primary finger is coupled directly to the primary drive element) are moved in the driving direction at the same rate as one another. For example, it may be that two or more (e.g. all) of the plurality of fingers are moved in the first direction by different amounts (e.g. through different distances or by different angular amounts) when the corresponding two or more (e.g. all) secondary drive elements (and optionally the primary drive element, in embodiments in which a primary finger is coupled directly to the primary drive element) are moved in the driving direction by the same amount (e.g. when the corresponding two or more secondary drive elements (and optionally the primary drive element) are displaced through the same distance in the driving direction) due to the difference in drive radii.

It may be that the coupling mechanisms are configured such that an outermost finger of the plurality fingers (e.g. corresponding to the little finger of a human hand) is moved in the first direction relative to the hand body at a faster rate than the other fingers when the secondary drive elements (and optionally the primary drive element, in embodiments in which a primary finger is coupled directly to the primary drive element) are moved in the driving direction at the same rate as one another. For example, it may be that the drive wheel of the outermost finger of the plurality of fingers has the smallest drive radius (i.e. of all the drive wheels present).

It may be that the coupling mechanisms are configured such that each finger of the plurality fingers is moved in the first direction relative to the hand body at a different rate than the other fingers when the secondary drive elements (and optionally the primary drive element, in embodiments in which a primary finger is coupled directly to the primary drive element) are moved in the driving direction at the same rate as one another. For example, it may be that each of the drive wheels has a different drive radius.

It may be that the rate at which each finger moves in the first direction relative to the hand body (i.e. when the secondary drive elements (and optionally the primary drive element, in embodiments in which a primary finger is coupled directly to the primary drive element) are moved in the driving direction at the same rate as one another) varies monotonically from one outermost finger to the other outermost finger. For example, it may be that the rate at which each finger moves in the first direction relative to the hand body (i.e. when the secondary drive elements (and optionally the primary drive element, in embodiments in which a primary finger is coupled directly to the primary drive element) are moved in the driving direction at the same rate as one another) decreases monotonically from the outermost finger corresponding to the pinkie finger to the other outermost finger corresponding to the index finger. For example, it may be the drive radii of the drive wheels vary monotonically from one outermost finger to the other outermost finger. For example, may be that the drive radii of the drive wheels increase monotonically from the outermost finger corresponding to the pinkie finger to the other outermost finger corresponding to the index finger.

In some examples, the prosthetic hand comprises one primary finger coupled directly to the primary drive element (i.e. not by way of a secondary drive element), three secondary fingers coupled to corresponding secondary drive elements, and a thumb. The primary finger corresponds to the index finger of a human hand and the three secondary fingers consist of fingers corresponding to the little finger, ring finger and middle finger of a human hand. The primary finger is coupled to the primary drive element by a primary finger coupling mechanism. The three secondary fingers are coupled to the corresponding secondary drive elements by corresponding secondary finger coupling mechanisms. The primary and secondary coupling mechanisms are configured such that the secondary finger corresponding to the pinkie finger is moved in the first direction relative to the hand body at a faster rate than the other fingers when the secondary drive elements and the primary drive element are moved in the driving direction at the same rate as one another. It may be that the rate at which each of the primary and secondary fingers moves in the first direction relative to the hand body (i.e. when the secondary drive elements and the primary drive element are moved in the driving direction at the same rate as one another) varies (e.g. increases) monotonically from the finger corresponding to the pinkie finger to the finger corresponding to the index finger.

The prosthetic hand may be a cable-operated prosthetic hand.

The prosthetic hand may comprise an operating cable coupled to the primary drive element and operable to move the primary drive element in the driving direction.

The operating cable may be coupled to the primary drive element in such a way that an operating force (i.e. tension) applied to the operating cable causes a driving force to be applied to the primary drive element which urges movement of the primary drive element in the driving direction. The operating cable may be further coupled to the primary drive element in such a way that the operating force is amplified, e.g. by the principle of mechanical advantage.

The operating cable may be coupled to the primary drive element by any suitable mechanical linkage(s) or kinematic chain(s). The mechanical linkage(s) or kinematic chain(s) may be configured to transmit, and optionally to amplify, the operating force (i.e. tension) applied to the operating cable to the primary drive element to urge movement of the primary drive element in the driving direction. For example, the operating cable may be coupled to the primary drive element by one or more pulleys, levers, cams, screws, gear chains, or joints, or any combination thereof.

The operating cable may be coupled to the primary drive element by one or more pulleys. The one or more pulleys may comprise one or more pulleys mounted on or forming part of the primary drive element and/or one or more pulleys mounted on the hand body. The use of one or more pulleys to drive movement of the primary drive element may enable amplification (by the principle of mechanical advantage) of the force applied to cause the movement of the primary drive element.

It may be that the primary drive element and the plurality of movable secondary drive elements are biased in a return direction which opposes the driving direction. For example, it may be that the driving direction is a direction pointing away from the plurality of fingers and the return direction is a direction pointing back towards the plurality of fingers.

Additionally or alternatively, it may be that the plurality of fingers are each biased in a second direction relative to the hand body which opposes the first direction. For example, it may be the first direction is a finger flexing direction and the second direction is a finger extending direction. That is to say, in some examples, movement of a finger in the first direction corresponds to flexing the finger and movement of the finger in the second direction corresponds to extending the finger.

The primary drive element and the plurality of movable secondary drive elements may be biased in the return direction and/or the plurality of fingers may be biased in the second direction by biasing means such as one or more springs or one or more elastic cords.

For example, it may be that the drive wheel of the coupling mechanism for each finger is coupled to the hand body by a corresponding return cable attached to, or comprising, a biasing means such as a spring or an elastic cord. In some examples, the drive wheel of the coupling mechanism for each finger is coupled to the hand body by a biasing means such as an elastic cord.

It may be that the coupling mechanism for each finger comprises a corresponding return wheel for driving movement of the said finger in the second direction relative to the hand body. It may be that each return wheel is coupled to the hand body by a corresponding return cable attached to, or comprising, a biasing means such as a spring or an elastic cord. In some examples, the return wheel of the coupling mechanism for each finger is coupled to the hand body by a biasing means such as an elastic cord.

It may be that the coupling mechanism for each finger comprises both a drive wheel and a return wheel, wherein the drive wheel and the return are distinct (le. different) components. Alternatively, it may be that the coupling mechanism for each finger comprises a single wheel which functions as both a drive wheel and a return wheel.

It may be that the primary drive element is coupled to the hand body by a biasing means such as a spring or an elastic cord. For example, the biasing means (e.g. spring or elastic cord) may be arranged such that movement of the primary drive element in the driving direction causes compression or extension of the biasing means (e.g. spring or elastic cord) in such a way which opposes the movement of the primary drive element in the driving direction and which causes movement of the primary drive element in the return direction when the force causing movement in the driving direction is released. The hand body may comprise a frame. The frame may support the mechanism for actuating movement of the plurality of fingers.

The one or more guides may comprise (e.g. be) one or more guide rods and/or one or more guide rails.

The one or more guides (e.g. one or more guide rods and/or one or more guide rails) may extend parallel to the driving direction. Movement of the primary drive element and the secondary drive elements may therefore be constrained to movement parallel to the driving direction.

One or more (e.g. each) of the primary and/or secondary drive elements may be mounted on the one or more guides (e.g. one or more guide rods and/or one or more guide rails). One or more (e.g. each) of the primary and/or secondary drive elements may be slidable along the one or more guides (e.g. one or more guide rods and/or one or more guide rails).

The one or more guides may be fixedly attached to the frame.

The primary drive element and the secondary drive elements may be mounted on different guides. For example, it may be that the primary drive element is mounted on one or more primary drive element guides and the secondary drive elements are mounted on corresponding secondary drive element guides.

The primary drive element may be mounted on one (i.e. single) primary drive element guide (e.g. primary drive element guide rail). The primary drive element guide (e.g. primary drive element guide rail) may be positioned at an end of the primary drive element. The primary drive element may be mounted on two or more primary drive element guides (e.g. primary drive element guide rails). Each of the two or more primary drive element guides (e.g. primary drive element guide rails) may be positioned at an end of the primary drive element. For example, the two or more primary drive element guides (e.g. primary drive element guide rails) may be positioned at the same end of the primary drive element or at different ends of the primary drive element (for example, at opposing ends of the primary drive element). In some examples, the primary drive element may be mounted on a first primary drive element guide (e.g. primary drive element guide rail) and a second primary drive element guide (e.g. primary drive element guide rail), wherein the first and second primary drive element guides are positioned at opposing ends of the primary drive element. The first and second primary drive element guides may be fixedly attached to the frame (for example, at opposing sides of the hand body).

A mechanism in which the primary drive element is mounted on two or more primary drive element guides may be more robust and movement of the primary drive element may be smoother, more stable and/or more consistent.

Each secondary drive element may be mounted on a corresponding secondary drive element guide (e.g. secondary drive element rod). Each secondary drive element guide (e.g. secondary drive element rod) may be fixedly attached to the frame. Each end of each secondary drive element guide (e.g. secondary drive element rod) may be fixedly attached to the frame (i.e. each secondary drive element guide (e.g. secondary drive element rod) may be fixedly attached to the frame at both of its ends). For example, each secondary drive element guide (e.g. secondary drive element rod) may extend completely across an internal cavity within the frame in which the mechanism for actuating movement of the plurality of fingers is housed.

Alternatively, each secondary drive element guide (e.g. secondary drive element rod) may be fixedly attached to the frame at only one end (i.e. only one end of the secondary drive element guide). The other end of the secondary drive element guide (e.g. secondary drive element rod) may not be attached to the frame. Each secondary drive element guide (e.g. secondary drive element rod) may extend or project into the internal cavity within the frame (i.e. in which the mechanism for actuating movement of the plurality of fingers is housed). Each secondary drive element guide (e.g. secondary drive element rod) may extend only partially across the internal cavity. This may reduce the amount of space within the cavity taken up by the secondary drive element components.

The hand body may comprise a housing. The housing may surround (e.g. cover) the mechanism for actuating movement of the plurality of fingers. The housing may comprise (e.g. form) the frame.

The primary drive element may take any suitable shape depending on the design of the prosthetic hand. For example, the primary drive element may be a bar shaped element (e.g. a bar) or the primary drive element may include a predominantly bar shaped portion. In some examples, the primary drive element is an L-shaped element or a U-shaped element. For example, the primary drive element may comprise a predominantly bar shaped body portion and one projecting portion at a first end of the predominantly bar shaped body portion (in an L-shaped element) or two projecting portions, each located at opposing ends of the predominantly bar shaped body portion (in a U-shaped element). The primary drive element may take any suitable shape depending on the design of the prosthetic hand. For example, each secondary drive element may be a bar or block shaped element (e.g. a bar or block).

Each pulley (e.g. each primary drive pulley and/or secondary drive pulley) may comprise a pulley wheel mounted on an axle.

The coupling cable may be held in tension against the primary and secondary drive pulleys. For example, the coupling cable may be fixed at each end, thus holding the coupling cable in tension. For example, the coupling cable may be fixed at each end to the primary drive element.

Each cable described herein (e.g. the coupling cable, any of the finger coupling cables, any of the drive cables, any of the return cables, any of the operating cables and/or the thumb cable) may be any suitable type of cable made of any suitable type of material. For example, each cable may be (as appropriate based on the requirements) a belt, a chain, a rope, a cord or a tape.

It may be that each of the plurality of fingers is removably mounted on the hand body. For example, it may be that each of the plurality of fingers is removably mounted on the hand body as described hereinbelow in connection with the fourth aspect of the invention. It may be that each of the plurality of fingers is removably mounted on the hand body by way of the corresponding coupling mechanism. For example, it may be that each of the plurality of fingers is removably mounted on (e.g. attached to) the drive wheel of the corresponding coupling mechanism.

Each of the plurality of fingers may be movable through an angular range of motion. The angular range of motion for each finger may be no greater than about 150°, for example, no greater than about 125°, or no greater than about 100°, or no greater than about 90° or no greater than about 80°, or no greater than about 70°. The angular range of motion for each finger may be no less than about 45°, for example, no less than about 50°, or no less than about 60°, or no less than about 70°. The angular range of motion for each finger may be from about 45° to about 150°, for example, from about 50° to about 125°, or from about 50° to about 100°, or from 50° to about 90°, or from about 60° to about 80°, for example, about 70°. It may be that each finger is attached to the hand body in such a way as to achieve the desired angular range of motion. For example, it may be that each finger is coupled to the corresponding drive element by a corresponding coupling mechanism which is configured to control the angular range of motion to within the desired range. For example, it may be that the drive wheel (for example, the shape of the drive wheel) in the coupling mechanism at least in part determines the angular range of motion. It may be that the angular range of motion is defined by the range of angles through which the finger may be pivoted about the axis of the drive wheel, i.e. by the range of angles through which the drive wheel is free to rotate about its axis.

In embodiments which comprise a thumb and a thumb mechanism, the thumb mechanism may be operable by the operating cable. The thumb mechanism may form part of the mechanism for moving the plurality of fingers.

The prosthetic hand may be a body-powered prosthetic hand. It may be that the prosthetic hand does not comprise any motors for driving movement of any components of the prosthetic hand.

Alternatively, the prosthetic hand may be an electrically powered prosthetic hand. For example, the prosthetic hand may comprise one or more motors for driving movement of one or more components of the prosthetic hand. For example, the prosthetic hand may comprise a motor for driving movement of the primary drive element and/or the operating cable.

The prosthetic hand may be configured for attachment to a user's socket. For example, the prosthetic hand may comprise a base configured for attachment to a user's socket. Attachment may be achieved by any means known in the art. For example, the prosthetic hand may be configured to attach to any commercially available sockets, which may make use of screw thread connections, spring-loaded plug and socket connections, etc.

According to a second aspect, there is provided a method of operating the prosthetic hand according to the first aspect. The prosthetic hand may have any features described hereinabove in relation to the first aspect.

The method may comprise applying a force to the primary drive element to cause movement of the primary drive element in the driving direction, wherein movement of the primary drive element in the driving direction drives movement of one or more of the secondary drive elements in the driving direction via the coupling cable, and wherein movement of said one or more of the secondary drive elements in the driving direction drives movement of the one or more corresponding fingers in the first direction relative to the hand body. It may be that applying the force to the primary drive element to cause movement of the primary drive element in the driving direction, thereby driving movement of one or more of the secondary drive elements in the driving direction via the coupling cable, drives movement of the one or more corresponding fingers in a flexing direction (i.e. towards a flexed finger configuration).

In embodiments in which the primary drive element and the plurality of movable secondary drive elements are biased in a return direction which opposes the driving direction and/or wherein the plurality of fingers are each biased in a second direction relative to the hand body which opposes the first direction, the method may comprise reducing or releasing the force applied to the primary drive element to permit movement of the primary drive element and the one or more of the secondary drive elements in the return direction and movement of the one or more corresponding fingers in the second direction relative to the hand body. It may be that reducing or releasing the force applied to the primary drive element to permit movement of the primary drive element and the one or more of the secondary drive elements in the return direction causes movement of the one or more corresponding fingers in an extending direction (i.e. towards an extended finger configuration).

The method may comprise moving two or more (e.g. all) of the fingers in the first direction relative to the hand body at different rates from one another.

According to a third aspect, there is provided a kit of parts for assembling the prosthetic hand according to the first aspect. The kit of parts comprises the hand body, the plurality of fingers and the mechanism for actuating movement of the plurality of fingers relative to the hand body.

The plurality of fingers may be attached to the hand body or they may be provided separate from hand body but attachable to (i.e. mountable on) the hand body.

The mechanism for actuating movement of the plurality of fingers may be provided installed in the hand body or it may be provided separate from the hand body but installable in the hand body.

The components of the mechanism for actuating movement of the plurality of fingers (i.e. the movable primary drive element, the one or more primary drive pulleys, the plurality of movable secondary drive elements, the corresponding secondary drive pulleys and the coupling cable) may be provided in an assembled or disassembled state.

The kit of parts may further comprise any other components of the prosthetic hand described hereinabove in relation to the first aspect, whether in an assembled or disassembled state.

According to a fourth aspect, there is provided a prosthetic hand comprising a hand body, a plurality of fingers removably mounted or mountable on the hand body, and a mechanism for actuating movement of the plurality of fingers relative to the hand body when the plurality of fingers are mounted on the hand body.

The prosthetic hand may have any features or components as described hereinabove in relation to the first aspect.

Because the plurality of fingers are removably mounted or mountable on the hand body, the fingers may be removed for repair or maintenance or replaced when damaged or to change finger functionality or design.

It may be that each finger is removably mounted or mountable on the hand body by a corresponding mounting mechanism. It may be that each finger is therefore individually removable or mountable on the hand body.

Each mounting mechanism may comprise an attachment element on (e.g. attached to or integrally formed with) the hand body and a corresponding attachment element on (e.g. attached to or integrally formed with) the corresponding finger. The attachment element on the hand body and the attachment element on the finger may be configured for releasably retaining the finger on the hand body when the finger is mounted on the hand body by the mounting mechanism.

In some examples, the attachment element on the hand body and the attachment element on the finger may be attached to one another by one or more fasteners, such as one or more screws, bolts, clips, latches or ties.

In some examples, each mounting mechanism may comprise a screw for attaching the attachment element on the hand body and the attachment element on the finger to one another.

In some examples, each mounting mechanism may comprise a latching element and a corresponding connecting element configured to engage one another to releasably retain the corresponding finger on the hand body when the finger is mounted on the hand body by the mounting mechanism. The latching element may be attached to the hand body and the connecting element may be located on the finger. The latching element may comprise a protrusion (e.g. a tooth, hook or other projection) and the connecting element may comprise a corresponding recess (e.g. a cut-out or indentation). The protrusion and the recess may be configured to engage one another to releasably retain the finger on the hand body when the finger is mounted on the hand body by the mounting mechanism. Mounting the finger on the hand body may comprise inserting the protrusion into the corresponding recess in the mounting mechanism.

It may be that each mounting mechanism comprises a corresponding release mechanism operable to release the corresponding finger from the hand body when the said finger is mounted on the hand body by the mounting mechanism.

In some examples, releasing the finger from the hand body may comprise releasing a fastener. For example, releasing the finger from the hand body may comprise releasing a screw which attaches the attachment element on the hand body and the attachment element on the finger to one another.

In some examples, releasing the finger from the hand body may comprise releasing the protrusion from the corresponding recess in the mounting mechanism.

It may be that each of the plurality of fingers is pivotably mountable on the hand body such that each finger is pivotable relative to the hand body when mounted on the hand body. The mechanism for actuating movement of the plurality of fingers relative to the hand body may be for actuating pivoting of the plurality of fingers relative to the hand body. Movement of each of the fingers may be achieved in any way and in any mode as described hereinabove in relation to the first aspect. For example, each of the plurality of fingers may be mountable on the hand body such that each finger can be flexed or extended as described hereinabove in relation to the first aspect.

The function of the mounting mechanism may correspond to that of a knuckle in a human hand. The mounting mechanism may therefore be known as, or form part of an assembly known as, a knuckle assembly.

The mechanism for actuating movement of the plurality of fingers relative to the hand body may comprise a plurality of movable drive elements. Each finger of the plurality of fingers, when mounted on the hand body, may be coupled to a corresponding drive element of the plurality of drive elements by a coupling mechanism such that movement of a drive element in a driving direction drives movement of the corresponding finger in a first direction relative to the hand body. Each finger may be removably mountable on the hand body via the coupling mechanism.

For example, as described hereinabove in relation to the first aspect, the mechanism for actuating movement of the plurality of fingers relative to the hand body may comprise a primary drive element and a plurality of secondary drive elements. Each finger of the plurality of fingers, when mounted on the hand body, may be coupled to a corresponding secondary drive element or may be coupled directly to the primary drive element by the corresponding coupling mechanism.

It may be that the coupling mechanism for each finger comprises a corresponding drive wheel for driving movement of the said finger in the first direction. It may be that each drive wheel is coupled to the corresponding drive element (e.g. the primary or secondary drive element) by a corresponding drive cable. It may be that each finger is rigidly attached to the corresponding drive wheel when mounted on the hand body, for example, by way of the corresponding mounting mechanism (e.g. the attachment elements and the one or more fasteners, or the latching element and the corresponding connecting element). It will be appreciated that a finger may be “rigidly attached” to a drive wheel in the sense that the finger is attached to the drive wheel such that the finger moves rigidly with the drive wheel as the drive wheel rotates. However, a finger which is “rigidly attached” to the drive wheel is not permanently attached to the drive wheel but remains instead removable from the drive wheel.

In some examples, the drive wheel and the attachment element of the hand form part of the same component. For example, the coupling mechanism for a finger may comprise a drive body which has a drive wheel portion and an attachment portion. The drive wheel portion may be configured to be mounted on a support on which the drive wheel portion is rotatable. The attachment portion may be configured for attachment to the attachment portion of a finger.

As described hereinabove in relation to the first aspect, it may be that the plurality of movable drive elements (e.g. the primary and secondary drive elements) are biased in a return direction which opposes the driving direction and/or the plurality of fingers, when mounted on the hand body, may each be biased in a second direction relative to the hand body which opposes the first direction.

It may be that the coupling mechanism for each finger comprises a corresponding return wheel for driving movement of the said finger in the second direction relative to the hand. It may be that each return wheel is coupled to the hand body by a corresponding return cable attached to a biasing means. It may be that each finger is rigidly attached to the corresponding return wheel when mounted on the hand body. Again, it will be appreciated that a finger may be “rigidly attached” to a return wheel in the sense that the finger is attached to the return wheel such that the finger moves rigidly with the return wheel as the drive wheel rotates. However, a finger which is “rigidly attached” to the return wheel is not permanently attached to the return wheel but remains instead removable from the return wheel.

The prosthetic hand may comprise at least three or at least four removably mounted or mountable fingers. For example, the prosthetic hand may comprise four removably mounted or mountable fingers. The removably mounted or mountable fingers may correspond to the pinkie finger, ring finger, middle finger and index finger of a human hand.

The prosthetic hand may comprise a thumb. The thumb may be removably mounted or mountable on the hand body. The prosthetic hand may comprise a thumb mechanism for actuating movement of the thumb.

The prosthetic hand may be a body-powered prosthetic hand. It may be that the prosthetic hand does not comprise any motors for driving movement of any components of the prosthetic hand.

Alternatively, the prosthetic hand may be an electrically powered prosthetic hand. For example, the prosthetic hand may comprise one or more motors for driving movement of one or more components of the prosthetic hand. For example, the prosthetic hand may comprise a motor for driving movement of the primary drive element and/or the operating cable.

The prosthetic hand may be configured for attachment to a user's socket. For example, the prosthetic hand may comprise base configured for attachment to a user's socket. Attachment may be achieved by any means known in the art. For example, the prosthetic hand may be configured to attach to any commercially available sockets, which may make use of screw thread connections, spring-loaded plug and socket connections, etc.

According to a fifth aspect, there is provided a method of using the prosthetic hand according to the fourth aspect.

The method may comprise removing (i.e. demounting) one or more of the plurality of fingers from the hand body and/or mounting one or more of the plurality fingers on the hand body.

The method may comprise: removing one or more of the plurality of fingers from the hand body; carrying out a maintenance operation (e.g. a cleaning and/or repair operation) on the one or more fingers; and re-mounting the one or more fingers on the hand body.

The method may comprise: removing one or more of the plurality of fingers from the hand body; and mounting one or more different (i.e. replacement) fingers on the hand body.

According to a sixth aspect, there is provided a kit of parts for assembling the prosthetic hand according to the fourth aspect. The kit of parts comprises the hand body, the plurality of fingers removably mountable on the hand body, and the mechanism for actuating movement of the plurality of fingers relative to the hand body.

The mechanism for actuating movement of the plurality of fingers may be provided Installed in the hand body or it may be provided separate from the hand body but installable in the hand body. The components of the mechanism for actuating movement of the plurality of fingers may be provided in an assembled or disassembled state.

The kit of parts may further comprise any other components of the prosthetic hand described hereinabove in relation to the first or fourth aspects, whether in an assembled or disassembled state.

The skilled person will appreciate that, except where mutually exclusive, a feature described in relation to any one of the above aspects may be applied mutatis mutandis to any other aspect. Furthermore, except where mutually exclusive, any feature described herein may be applied to any aspect and/or combined with any other feature described herein.

For the avoidance of doubt, the application extends to the subject-matter set out in the following numbered paragraphs:
1. A prosthetic hand comprising a hand body, a plurality of fingers and a mechanism for actuating movement of the plurality of fingers relative to the hand body, wherein the mechanism comprises:
   a movable primary drive element comprising one or more primary drive pulleys; and
   a plurality of movable secondary drive elements each comprising a corresponding secondary drive pulley;
   wherein:
   each of the plurality of secondary drive elements is coupled to the primary drive element by a coupling cable supported by the primary and secondary drive pulleys, such that movement of the primary drive element in a driving direction away from the secondary drive elements exerts a force on the secondary drive elements urging movement of the secondary drive elements in the driving direction;
   each of the plurality of secondary drive elements is coupled to a corresponding finger of the plurality of fingers such that movement of a secondary drive element in the driving direction drives movement of the corresponding finger in a first direction relative to the hand body; and
   the primary drive element and the secondary drive elements are mounted on one or more guides which constrain the direction of movement of the said primary drive element and the secondary drive elements.
2. The prosthetic hand according to paragraph 1, wherein each finger of the plurality of fingers which is coupled to a secondary drive element is coupled to the corresponding secondary drive element by a corresponding coupling mechanism configured such that that two or more of the plurality of fingers are moved in the first direction relative to the hand body at different rates from one another when the corresponding two or more secondary drive elements are moved in the driving direction at the same rate as one another.
3. The prosthetic hand according to paragraph 2, wherein the coupling mechanism for each finger comprises a corresponding drive wheel for driving movement of the said finger in the first direction, wherein each drive wheel is coupled to the corresponding secondary drive element by a corresponding drive cable, and wherein two or more of the drive wheels have different drive radii such that the corresponding two or more fingers are moved in the first direction relative to the hand body at different rates from one another when the corresponding two or more secondary drive elements are moved in the driving direction at the same rate as one another.
4. The prosthetic hand according to paragraph 3, wherein the drive wheel of an outermost finger of the plurality of fingers has the smallest drive radius.
5. The prosthetic hand according to paragraph 4, wherein the outermost finger corresponds to a little finger.
6. The prosthetic hand according to any preceding paragraph, wherein the plurality of fingers is a plurality of secondary fingers and the prosthetic hand further comprises a primary finger coupled directly to the primary drive element such that movement of the primary drive element in the driving direction drives movement of the primary finger in the first direction relative to the hand body.
7. The prosthetic hand according to paragraph 6, wherein the primary finger corresponds to an index finger.
8. The prosthetic hand according to any preceding paragraph, wherein the prosthetic hand comprises an operating cable coupled to the primary drive element and operable to move the primary drive element in the driving direction.
9. The prosthetic hand according to paragraph 8, wherein the operating cable is coupled to the primary drive element by one or more pulleys, levers, cams, screws, gear chains or joints, or any combination thereof.
10. The prosthetic hand according to any preceding paragraph, wherein the primary drive element and the plurality of movable secondary drive elements are biased in a return direction which opposes the driving direction and/or wherein the plurality of fingers are each biased in a second direction relative to the hand body which opposes the first direction.
11. The prosthetic hand according to any preceding paragraph, wherein the plurality of fingers are removably mounted on the hand body.
12. The prosthetic hand according to any preceding paragraph, wherein the prosthetic hand further comprises a thumb and, optionally, a thumb mechanism for actuating movement of the thumb.
13. The prosthetic hand according to any preceding paragraph, wherein the prosthetic hand is a body-powered prosthetic hand.
14. A method of operating the prosthetic hand according to any preceding paragraph, wherein the method comprises applying a force to the primary drive element to cause movement of the primary drive element in the driving direction, wherein movement of the primary drive element in the driving direction drives movement of one or more of the secondary drive elements in the driving direction via the coupling cable, and wherein movement of said one or more of the secondary drive elements in the driving direction drives movement of the one or more corresponding fingers in the first direction relative to the hand body.
15. The method according to paragraph 14, wherein the primary drive element and the plurality of movable secondary drive elements are biased in a return direction which opposes the driving direction and/or wherein the plurality of fingers are each biased in a second direction relative to the hand body which opposes the first direction, and wherein the method comprises reducing or releasing the force applied to the primary drive element to permit movement of the primary drive element and the one or more of the secondary drive elements in the return direction and movement of the one or more corresponding fingers in the second direction relative to the hand body.
16. A prosthetic hand comprising a hand body, a plurality of fingers removably mounted or mountable on the hand body, and a mechanism for actuating movement of the plurality of fingers relative to the hand body when the plurality of fingers are mounted on the hand body.
17. The prosthetic hand according to paragraph 17, wherein each finger is removably mounted or mountable on the hand body by a corresponding mounting mechanism which comprises a latching element attached to the hand body and a corresponding connecting element on the finger, wherein the latching element and the connecting element are configured to engage one another to releasably retain the finger on the hand body when the finger is mounted on the hand body by the mounting mechanism.
18. The prosthetic hand according to paragraph 16, wherein each finger is removably mounted or mountable on the hand body by a corresponding mounting mechanism which comprises an attachment element on the hand body, a corresponding attachment element on the finger, and one or more fasteners for attaching the attachment element on the hand body and the attachment element on the finger to one another.
19. The prosthetic hand according to paragraph 16 or paragraph 17, wherein each mounting mechanism comprises a corresponding release mechanism operable to release the corresponding finger from the hand body when the said finger is mounted on the hand body by the mounting mechanism.
20. The prosthetic hand according to any of paragraphs 16 to 19, wherein each of the plurality of fingers is pivotably mountable on the hand body such that each finger is pivotable relative to the hand body when mounted on the hand body, and wherein the mechanism for actuating movement of the plurality of fingers relative to the hand body is for actuating pivoting of the plurality of fingers relative to the hand body.
21. The prosthetic hand according to any of paragraphs 16 to 20, wherein the mechanism for actuating movement of the plurality of fingers relative to the hand body comprises a plurality of movable drive elements, wherein each finger of the plurality of fingers, when mounted on the hand body, is coupled to a corresponding drive element of the plurality of drive elements by a coupling mechanism such that movement of a drive element in a driving direction drives movement of the corresponding finger in a first direction relative to the hand body, and wherein each finger is removably mountable on the hand body via the coupling mechanism.
22. The prosthetic hand according to paragraph 21, wherein the coupling mechanism for each finger comprises a corresponding drive wheel for driving movement of the said finger in the first direction, wherein each drive wheel is coupled to the corresponding drive element by a corresponding drive cable, and wherein each finger is rigidly attached to the corresponding drive wheel when mounted on the hand body.
23. The prosthetic hand according to paragraph 22, wherein the plurality of movable drive elements are biased in a return direction which opposes the driving direction and/or wherein the plurality of fingers, when mounted on the hand body, are each biased in a second direction relative to the hand body which opposes the first direction.
24. The prosthetic hand according to paragraph 23, wherein the prosthetic hand is a body-powered prosthetic hand.

### FIGURES

Embodiments will now be described by way of example only, with reference to the Figures, in which:

FIG. 1 is a perspective view of a first example prosthetic hand;

FIG. 2 is an exploded view of part of the prosthetic hand of FIG. 1;

FIG. 3 is another exploded view of part of the prosthetic hand of FIG. 1;

FIG. 4 is an exploded view of a finger slider assembly for the prosthetic hand of FIG. 1;

FIG. 5 is a perspective view of a drive bar assembly for the prosthetic hand of FIG. 1;

FIG. 6 is an exploded view of the drive bar assembly for the prosthetic hand of FIG. 1;

FIG. 7 is an exploded view of a knuckle assembly for the prosthetic hand of FIG. 1;

FIG. 8 is (a) a perspective and (b) an exploded view of part of the knuckle assembly for the prosthetic hand of FIG. 1;

FIG. 9 illustrates three different configurations (a), (b) and (c) of a mechanism for moving the fingers of the prosthetic hand of FIG. 1;

FIG. 10 illustrates two different configurations (a) and (b) of a control mechanism of the prosthetic hand of FIG. 1;

FIG. 11 illustrates a return mechanism for moving a finger of the prosthetic hand of FIG. 1;

FIG. 12 illustrates a drive mechanism for moving a finger of the prosthetic hand of FIG. 1;

FIG. 13 illustrates two different finger positions (a-extended) and (b-flexed) in perspective and side views for the prosthetic hand of FIG. 1;

FIG. 14 illustrates a finger latching mechanism of the prosthetic hand of Figure

FIG. 15 illustrates the finger latching mechanism in more detail, where (a) shows a finger in an unlatched configuration in top view and cross-section B-B, (b) shows the finger in a latched configuration in top view and cross-section C-C, (c) provides a closer view of the latching mechanism in (a), and (d) provides a closer view of the latching mechanism in (b);

FIG. 16 is a perspective view of a second example prosthetic hand;

FIG. 17 is an exploded view of the prosthetic hand of FIG. 16;

FIG. 18 is a perspective view of a chassis assembly of the prosthetic hand of FIG. 16;

FIG. 19 is an exploded view of the chassis assembly of FIG. 18;

FIG. 20 is a perspective view of a knuckle assembly of the prosthetic hand of FIG. 16;

FIG. 21 is an exploded view of the knuckle assembly of FIG. 20;

FIG. 22 illustrates three different configurations (a), (b) and (c) of a mechanism for moving the fingers of the prosthetic hand of FIG. 16;

FIG. 23 illustrates a configuration of a control mechanism of the prosthetic hand of FIG. 16;

FIG. 24 illustrates four different finger drive wheels (a), (b), (c) and (d) of the prosthetic hand of FIG. 16;

FIG. 25 illustrates a finger attachment mechanism of the prosthetic hand of FIG. 16, wherein a finger is in (a) a detached configuration and (b) an attached configuration;

FIG. 26 illustrates the finger attachment mechanism of FIG. 25 in more detail; and

FIG. 27 illustrates a knuckle drive body (a) in side view and (b) in cross-section along D-D when mounted between knuckle core plates.

### DETAILED DESCRIPTION

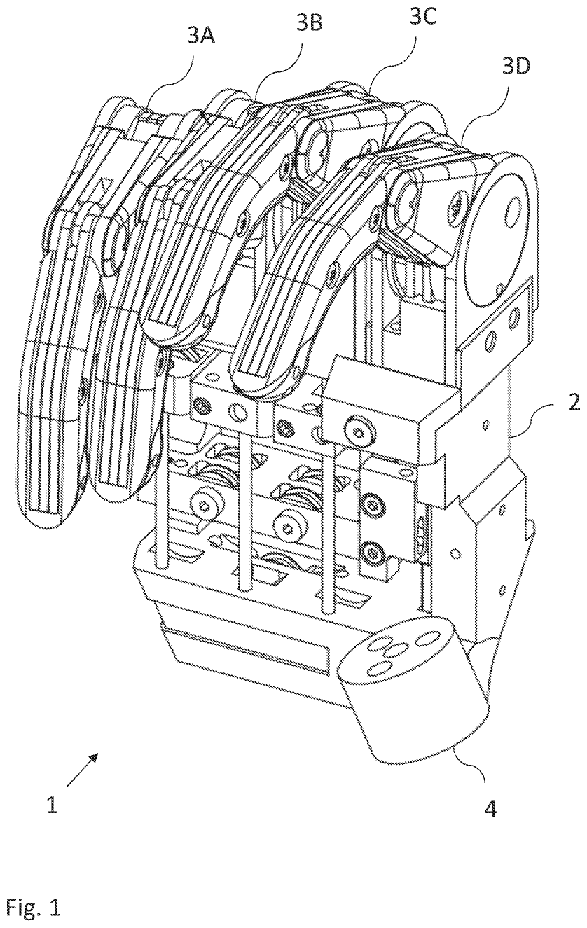

With reference to FIG. 1, a first example prosthetic hand 1 includes a hand body 2, four fingers 3A, 3B, 3C and 3D and a thumb mount 4. The prosthetic hand 1 is configured to be mounted onto a user's socket. Finger 3A corresponds to the pinkie or little finger of a human hand. Finger 3B corresponds to the ring finger of a human hand. Finger 3C corresponds to the middle finger of a human hand. Finger 3D corresponds to the index finger of a human hand.

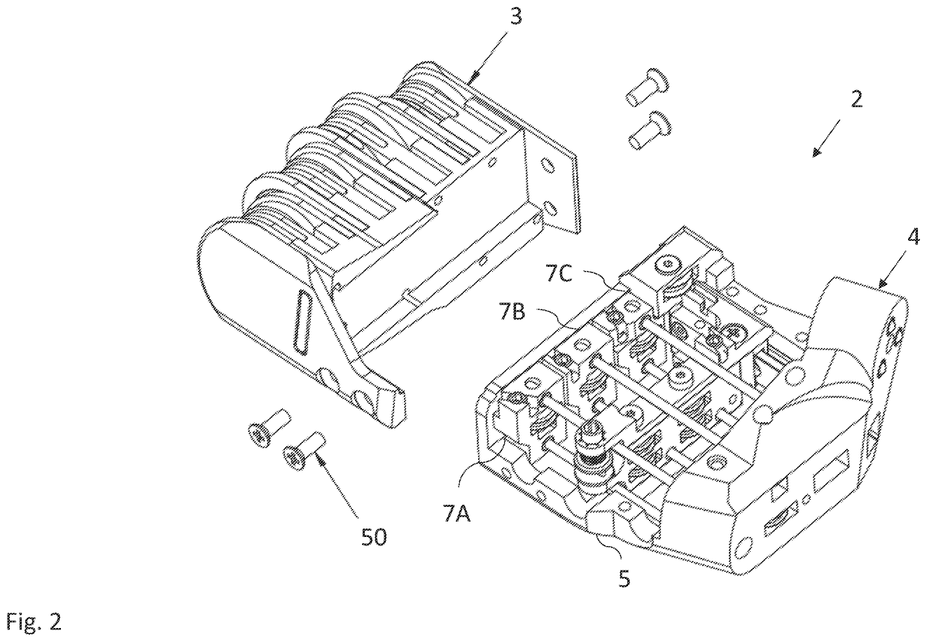

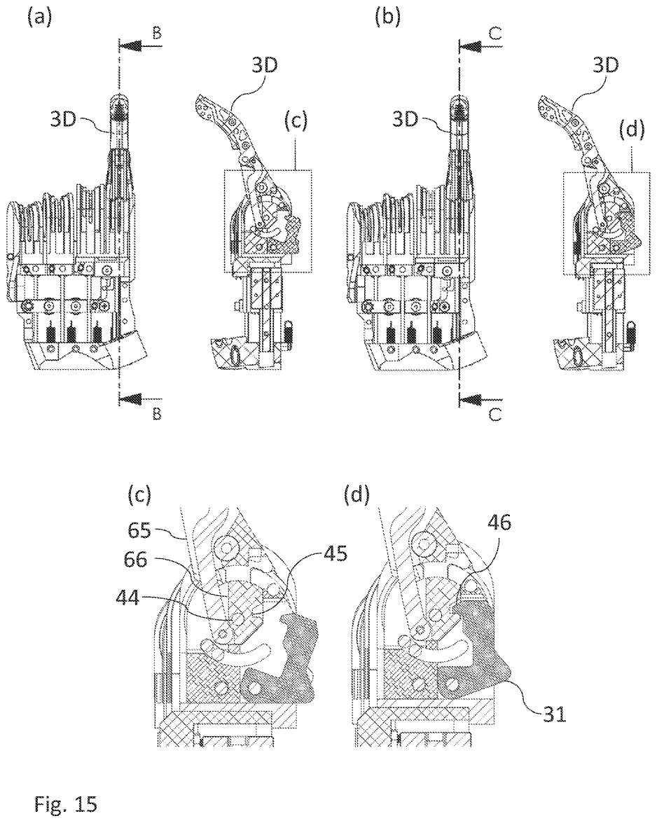

The prosthetic hand 1 is shown in more detail in FIGS. 2 to 15.

As shown in FIG. 2, the hand 1 includes a hand body 2 and a knuckle body 3. Knuckle body 3 is fixedly mountable on the hand body 2 by way of screws 50. Knuckle body 3 includes knuckle assemblies for supporting fingers 3A-3D, as discussed in more detail hereinbelow.

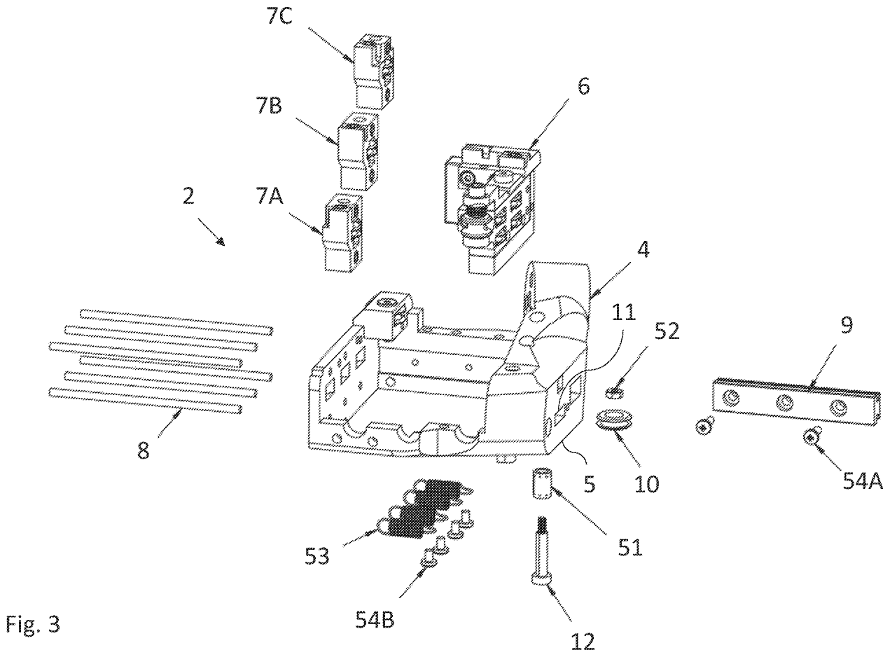

As shown in FIG. 3, the hand body 2 includes a support frame (or housing) 5 which supports a mechanism for controlling movement of the fingers 3A-3D. The mechanism includes a drive bar assembly (a primary drive element) 6 and finger slider assemblies (secondary drive elements) 7A, 7B and 7C which are slidably mountable within the frame 5 on alignment rods 8 and drive bar rail 9 (which function as guides). Drive bar rail 9 is fixed to the support frame 5 by pan head screws 54A. An amplifier pulley wheel 10 is mounted in a base portion 11 of the frame 5 by shoulder screw 12, needle bearing 51, and hex nut 52. Return springs 53 are mounted within the frame 5 and attached at one end to pan head screws 54B.

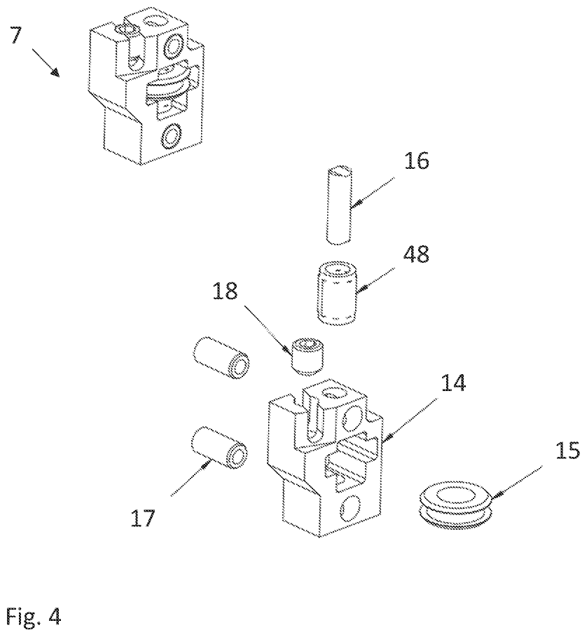

As shown in FIG. 4, each finger slider assembly 7 includes a slider body 14 within which a pulley wheel 15 is mounted on a pulley axle 16 surrounded by a needle bearing 48. The finger slider assembly 7 also comprises slide bearings 17 for providing a sliding interface with the alignment rods 8. The finger slider assembly 7 also includes a set screw 18 for adjusting the tension on a cable to be supported by the pulley 15 (as discussed in more detail hereinbelow).

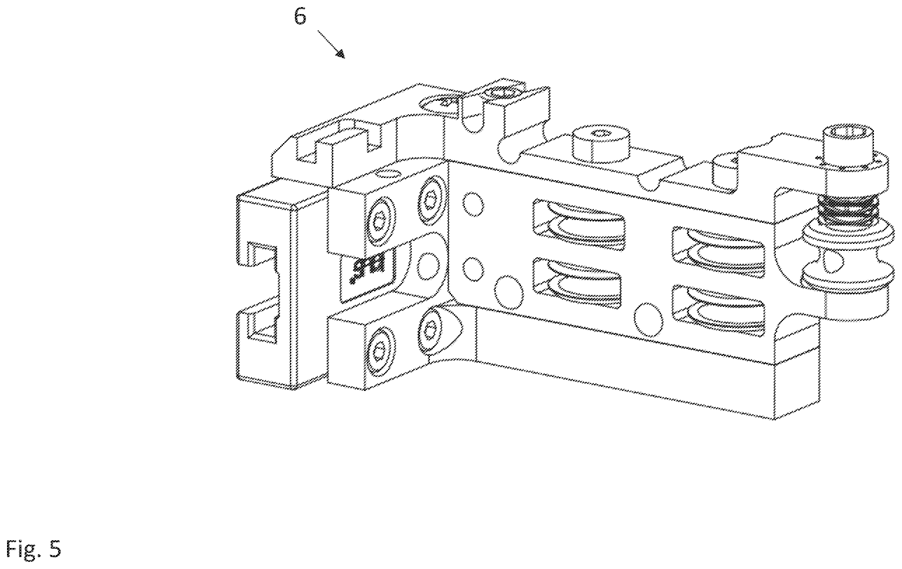

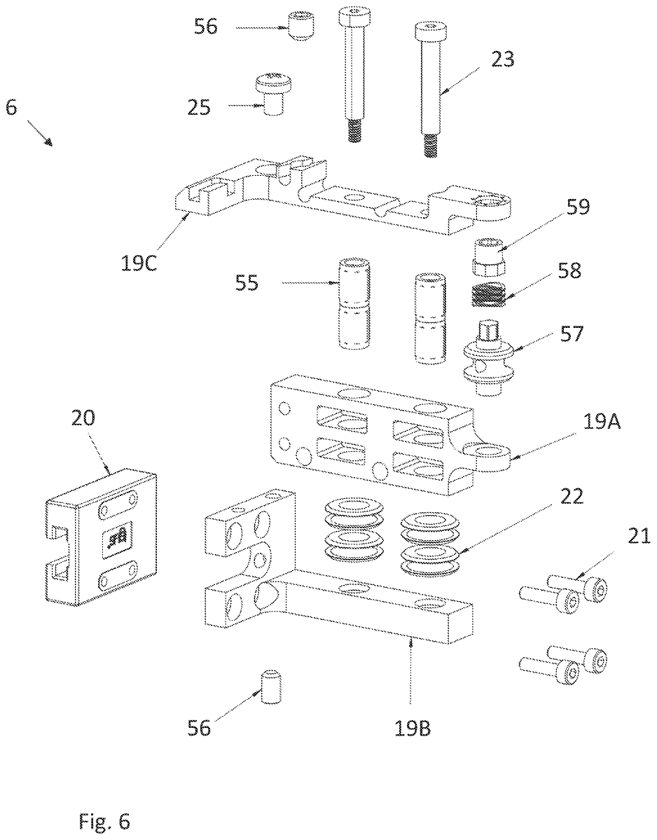

As shown in FIGS. 5 and 6, the drive bar assembly 6 includes a drive bar sheath 19A attached to a drive bar base 19B by a drive bar cap 19C, together forming a drive bar 19, which assembly is itself attached to a drive rail carriage 20 by screws 21 for slidably mounting the drive bar 19 on the drive bar rail 9. Four pulley wheels 22 are mounted on pulley axle screws 23 with corresponding needle bearings 55 within the drive bar sheath 19A. The drive bar sheath 19A, drive bar base 19B and drive bar cap 19C are held together by axle screws 23 and pan head screw 25. A tension barrel 57, together with wave spring 58 and tension cap 59, are mounted in a corresponding aperture at one end of the drive bar sheath 19A.

FIG. 2 illustrates the position of the drive bar assembly 6 and finger slider assemblies 7A, 7B and 7C within the frame 5 when slidably mounted on the rods 8 and rail 9.

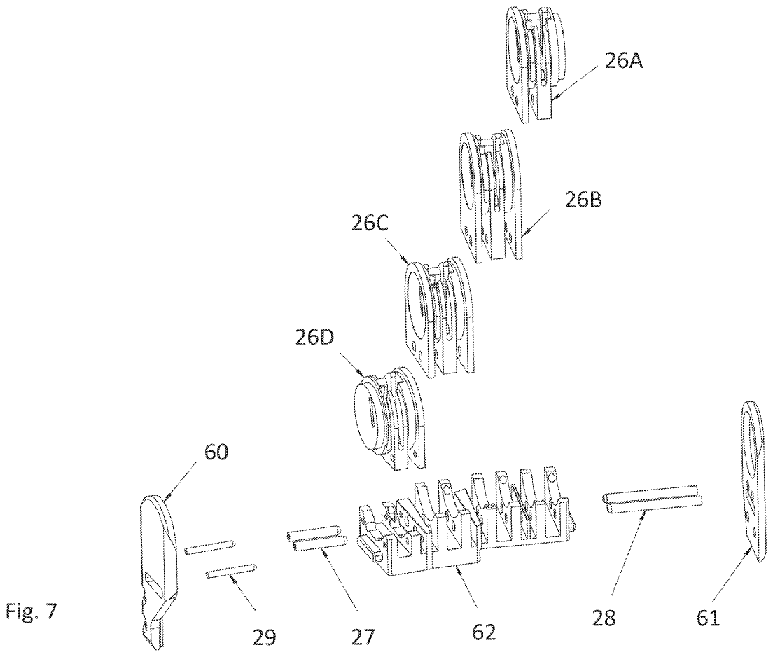

FIG. 7 shows the knuckle body 3 in more detail. The knuckle body 3 includes four knuckle assemblies 26A, 26B, 26C and 26D which are rigidly mountable on the frame 5 by way of cap members 60 and 61, knuckle mount bodies 62, and lock pins 27, 28 and 29.

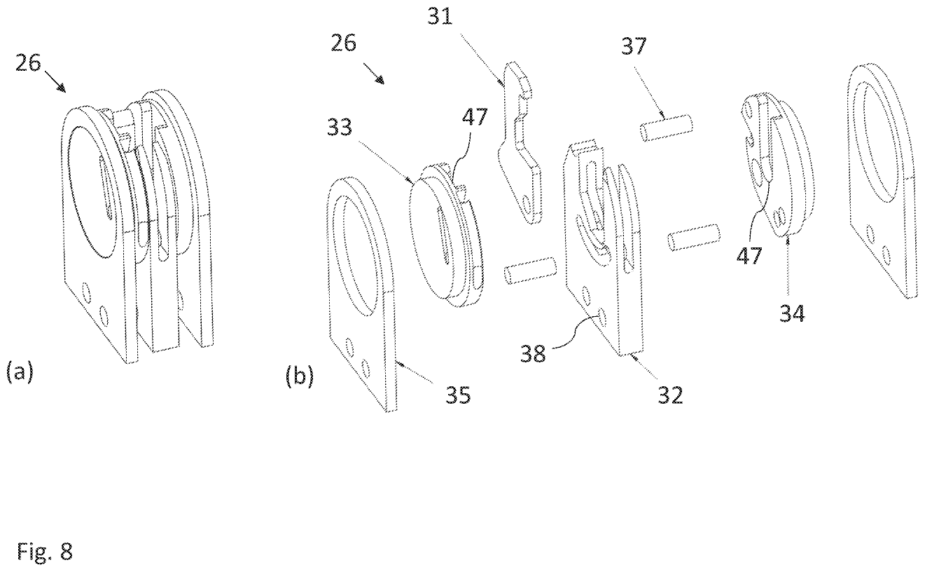

As shown in FIG. 8, each knuckle assembly 26 includes a finger latch 31 positioned within a knuckle core 32 and sandwiched between a drive wheel 33 and a return wheel 34 positioned either side of the knuckle core, and two exterior knuckle plates 35 configured to provide bearing surfaces for the drive and return wheels 33 and 34. The finger latch 31 is configured to connect to one of the fingers 3A-3D, as described in more detail hereinbelow. Spacer pins 37 connect the drive wheel 33 and return wheel 34 together and maintain alignment therebetween. The knuckle plates 35 and knuckle core 32 comprise apertures 38 through which they are mountable on the knuckle mount bodies 62 by corresponding lock pins 27, 28 and/or 29.

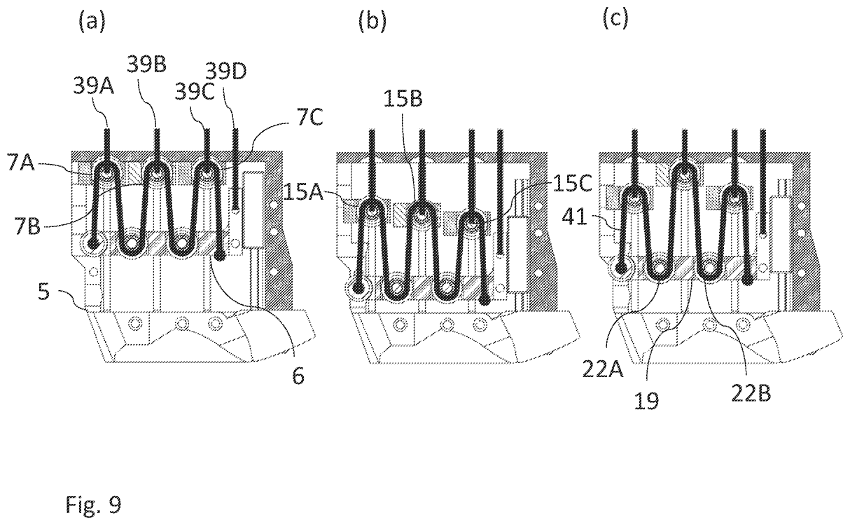

The prosthetic hand 1 is a cable-operated prosthetic hand. Thus, as illustrated in FIGS. 9 to 12, various components of the mechanism for controlling movement of the fingers are operably coupled to one another by way of cables.

As shown in FIGS. 9, 11 and 12, each of knuckle assemblies 26A, 26B and 26C is coupled to a corresponding finger slider assembly 7A, 7B and 7C by corresponding drive cables 39A, 39B and 39C and to corresponding return springs 53A, 53B and 53C by corresponding return cables 40A, 40B and 40C (for simplicity, FIG. 9 only shows drive cables 39A-39C). Knuckle assembly 26D is coupled directly to the drive bar assembly 6 by corresponding drive cable 39D attached to the drive bar cap 19C and to return spring 53D by return cable 40D (for simplicity, FIG. 9 only shows drive cable 39D). In addition, the finger slider assemblies 7A, 7B and 7C are coupled to the drive bar assembly 6 by a coupling cable 41 supported by the pulley wheels 15A, 15B, 15C, 22A and 22B and fixed at each end to the drive bar 19 (at one end via the tension barrel 57 for applying tension to the coupling cable 41).

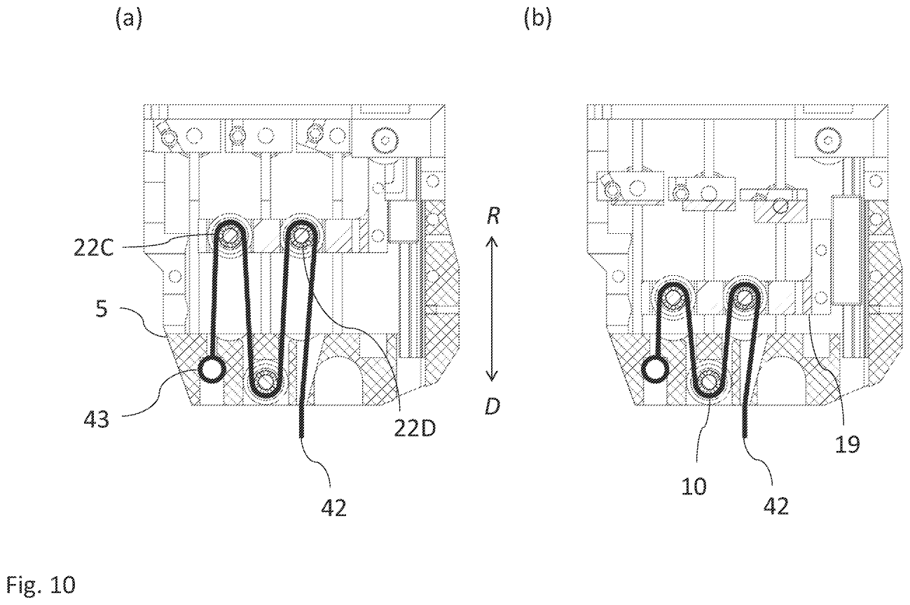

As shown in FIG. 10, the drive bar 19 is also coupled to the frame 5 by an operating cable 42 supported by the pulley wheels 10, 22C and 22D and fixed at one end 43 to the frame 5. For simplicity and to illustrate the two mechanisms more clearly, the operating cable 42 is not illustrated in FIG. 9 and the driving, return and coupling cables 39, 40 and 41 are not illustrated in FIG. 10.

When the prosthetic hand 1 is assembled, the hand is operable to provide control of movement of the fingers 3A to 3D via the corresponding knuckle assemblies 26A, 26B, 26C and 26D by applying tension to, or releasing tension from, operating cable 42.

In particular, when tension is applied to operating cable 42, a force is exerted on the drive bar assembly 6 urging movement of the drive bar assembly 6 in a driving direction D indicated in FIG. 10. When the drive bar assembly 6 is free to move in the driving direction D, it will slide along the rods 8 and rail 9 away from the knuckle assemblies towards the base of the frame 5. When the tension applied to operating cable 42 is released, the return force exerted on the drive bar assembly 6 by the spring units 53A to 53D will urge movement of the drive bar assembly 6 in a return direction R also indicated in FIG. 10. When the drive bar assembly 6 is free to move in the return direction R, it will slide along the rods 8 and rail 9 away from the base of the frame 5 back towards the knuckle assemblies. Thus, application and release of tension to the operating cable 42 can be used to control movement of the drive bar assembly 6 (guided by the rods 8 and rail 9).

FIG. 10 (a) illustrates a configuration of the prosthetic hand 1 in which the drive bar assembly 6 has travelled as far as possible in return direction R. FIG. 10 (b) illustrates a configuration of the prosthetic hand 1 in which the drive bar assembly 6 has travelled as far as possible in the driving direction D.

As illustrated in FIG. 9, the drive bar assembly 6 moves rigidly with the drive bar assembly 6. By controlling the movement of the drive bar assembly 6, tension can therefore be applied directly to or released from the finger drive cable 39D and the finger return cable 40D.

In addition, movement of the drive bar assembly 6 can drive movement of the finger slider assemblies 7A, 7B and 7C. In particular, when the drive bar assembly 6 moves in the driving direction D, a force is applied to each of the slider assemblies 7A, 7B and 7C, through the coupling cable 41, which urges movement of each of the slider assemblies 7A, 7B and 7C in the driving direction D. When one or more of the slider assemblies are free to move in the driving direction D, the said one or more slider assemblies will slide along the rods 8 in the driving direction under the applied force. When the drive bar assembly 6 moves in the return direction R, the force acting on the slider assemblies 7A, 7B and 7C is released and the return force exerted on the slider assemblies 7A, 7B and 7C by the spring unit will urge movement of the slider assemblies 7A, 7B and 7C in the return direction R. When one or more of the slider assemblies are free to move in the return direction R, the said one or more slider assemblies will slide along the rods 8 in the return direction under the return force.

Thus, application and release of tension to the operating cable 42 can be used to control movement of the slider assemblies 7A, 7B and 7C (guided by the rods 8). Moreover, although controlled by application and release of tension to a single operating cable 42, slider assemblies 7A, 7B and 7C are movable individually from one another, at least to a certain extent. In particular, if one of the slider assemblies is not free to move in the direction D (for example, because the finger controlled by said slider assembly is in contact with an object, as discussed in more detail hereinbelow), the remaining slider assemblies which are free to move in the direction D will still move in the direction D when the drive bar assembly is pulled in direction D, while the slider assembly which is not free to move will remain stationary. The prosthetic hand 1 therefore provides for differential control of movement of the slider assemblies 7A, 7B and 7C.

FIG. 9 (a) illustrates a configuration of the prosthetic hand 1 in which the drive bar assembly 6 has travelled as far as possible in return direction R and thus all three finger slider assemblies 7A, 7B and 7C have also travelled as far as possible in return direction R. FIGS. 9 (b) and (c) illustrate different configurations of the prosthetic hand 1 in which the finger slider assemblies 7A, 7B and 7C have been pulled different distances in driving direction D by the drive bar assembly 6.

When the slider assemblies 7A, 7B or 7C and/or the drive bar assembly 6 move, they apply tension to or release tension from the corresponding drive cables 39A, 39B, 39C and 39D and return cables 40A, 40B, 40C and 40D, through which movement of the corresponding fingers 3A, 3B, 3C and 3D can be achieved.

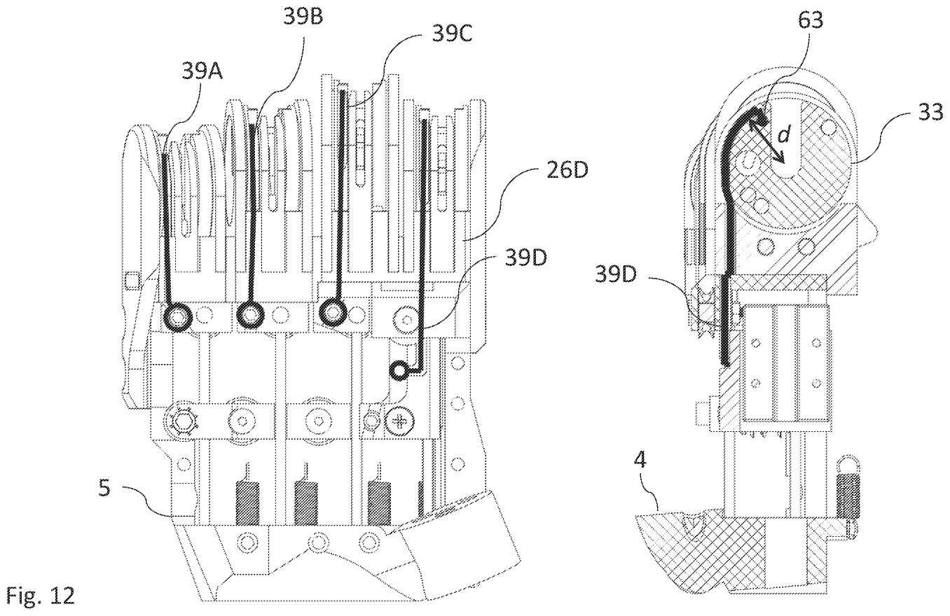

In particular, as shown in FIG. 12, each drive cable 39 is attached to the drive wheel 33 of the corresponding knuckle assembly 26 at attachment point 63. When tension is applied to the drive cable 39, a torque is applied to the drive wheel 33 which urges rotation of the drive wheel 33 about its axis in a first sense. When a finger is attached to the knuckle assembly 26 by the finger latch 31 (as described hereinbelow), rotation of the drive wheel 33 in the first sense drives movement of the finger in a first direction (if the finger is free to move in the first direction).

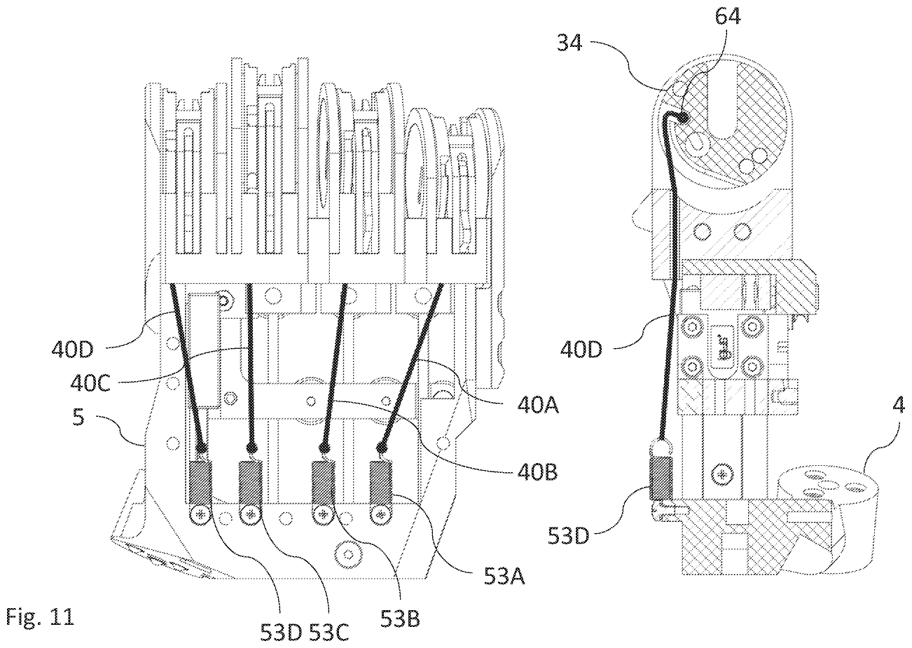

Similarly, when tension applied to the drive cable 39 is released, tension applied to the return wheel 34 by the return cable 40 (which is attached to the return wheel at attachment point 64, as shown in FIG. 11) causes a torque to be applied to the return wheel 34 which urges rotation of the return wheel 34 about its axis in a second sense opposing the first sense. When a finger is attached to the knuckle assembly 26 by the finger latch 31, rotation of the return wheel 34 in the second sense drives movement of the finger in a second direction opposing the first direction (if the finger is free to move in the second direction).

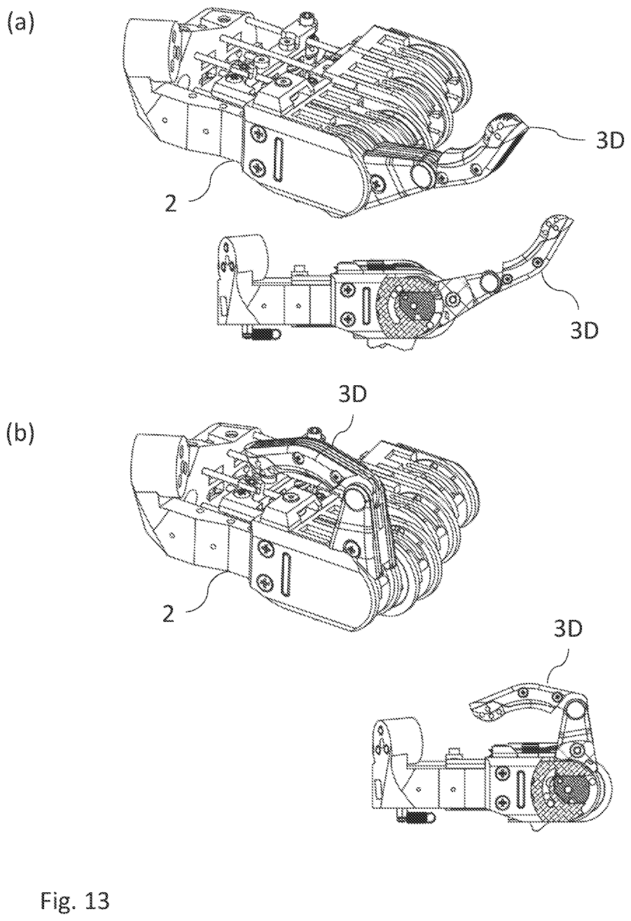

The mechanisms for attaching fingers 3A-3D to knuckle assemblies 26A-26D and moving said fingers 3A-3D are illustrated in more detail in FIGS. 13 to 15. Each finger (only finger 3D is shown in FIGS. 13 to 15 for simplicity) comprises a finger body 65 pivotally coupled to a connecting element 66 by links 67 and 68 at a knuckle (i.e. proximal) end of the finger. The connecting element 66 includes a cutout portion 45 configured to engage with a tooth-shaped protrusion 46 on the finger latch 31. The finger can be attached to the hand by sliding the knuckle end of the finger into the corresponding knuckle assembly 26 such that a central axle 44 slides into the corresponding slot 47 on each of the drive and return wheels 33 and 34 (thus aligning the central axle 44 with the axis of rotation for the drive and return wheels 33 and 34) and engaging the tooth 46 with the cutout portion 45, thus locking the finger in place.

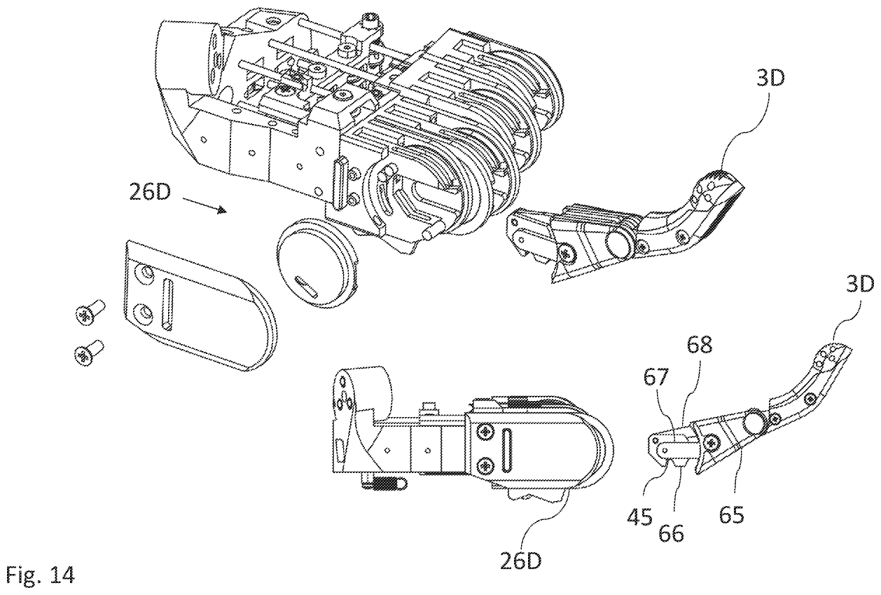

The finger latch 31 and the connecting element 66 are typically configured to provide a strong “snap fit” connection between the components. However, the latch mechanism is easily releasable by pivoting the finger latch 31 out of position such that the tooth 46 disengages the cutout portion 45. The finger is therefore easily removable from the hand for cleaning, maintenance or replacement, as illustrated in FIG. 14. The finger may be easily installed or removed even using only one hand (e.g. the user's free hand).

It will be appreciated that, once a finger is locked into place in a corresponding knuckle assembly, the finger is movable relative to the hand body 5 by applying or releasing tension to the corresponding drive cable (as discussed hereinabove). As the drive wheel 33 and return wheel 34 rotate, they press against corresponding finger components (e.g. links 67), driving pivoting of the finger body 65 relative to the connecting element 66 and thus also relative to the hand body 5. The direction of motion will depend on the shape of the finger and the design and placement of the knuckle assembly. However, in the example shown, the finger is configured to be movable in a flexion-extension mode of movement, permitting movement of the finger between an extended configuration (in which a distal end of the finger is furthest from the palm of the hand, as illustrated in FIG. 13 (a)) and a flexed configuration (in which the distal end of the finger is closest to the palm of the hand, as illustrated in FIG. 13 (b)).

In the embodiment shown in the figures, the drive wheels 33A, 33B, 33C and 33D of each of the knuckle assemblies 26A, 26B, 26C and 26D are not all identical and differ in terms of drive radii d. As can be seen in FIG. 12, the drive radius d of a given drive wheel is defined as the shortest straight line distance between the central axis of the wheel (about which the wheel rotates in the knuckle assembly) and the circumferential drive surface of the drive wheel against which the drive cable rests. It will be appreciated that, as the drive radius for a given drive wheel is reduced, the angular rotation of the drive wheel about its axis increases for a given drive cable displacement. Accordingly, when the drive radii of the drive wheels are different, all of the fingers are free to move, and tension is applied to the operating cable 42 to cause the drive bar assembly 6 and the finger slider assemblies 7A, 7B and 7C to move towards the base of the hand frame 5 at the same rate (such that the drive bar assembly 6 and the finger slider assemblies 7A, 7B and 7C are simultaneously displaced through the same distance), the fingers will be rotated about their respective drive wheel axes at different (i.e. angular) rates.

Therefore, where the fingers and knuckle assemblies are configured such that applying tension to the drive cables urges rotation of the drive wheels to cause flexing movement of the corresponding fingers towards the palm of the hand body, then, if all four of the fingers are free to move, the fingers will flex towards the palm at different rates. It will be appreciated that the drive radii of the different drive wheels can be selected to target different or the same relative rates of movement. For example, it may be desirable that all or some of the fingers move at different rates, or that all or some of the fingers move at the same rate. It will be appreciated that different types of concerted movement of the fingers may be useful for different types of task. For example, concerted movement of the fingers in which the rate of flexing increases going from the index finger to the pinkie finger (i.e. such that the pinkie finger flexes most quickly) may be particularly useful when the prosthetic hand is used to grasp an object such as a glass or a cup.

Moreover, because of use of the finger slider assemblies 7A, 7B and 7C, the remaining fingers of 3A, 3B and 3C remain moveable (for example, in a flexing direction) even if movement of one or two of fingers 3A, 3B and 3C is blocked, for example, because these fingers have already come into contact with an object. This enables the grip achieved using the prosthetic hand to adapt to (e.g. mould around) different object shapes.

In addition, because drive cable 3D is connected directly to the drive bar assembly 6 rather than to a corresponding finger slider assembly coupled to the other finger slider assemblies by the coupling cable 41, movement of finger 3D is controlled directly by movement of the drive bar assembly 6. This enables significantly more force to be directed through finger 3D, replicating the index finger pinching force which is typically stronger in human hands.

Staggered flexing of the fingers through use of drive wheels 33 having different drive radii mitigates a potential downside of having the index finger 3D coupled directly to the drive bar assembly 6. In particular, since the index finger 3D is directly coupled to drive bar assembly 6, motion of the drive bar assembly 6 (and thus also of any of the finger sliding assemblies) in the driving direction D is not possible once movement of the index finger 3D is blocked (for example, because the index finger 3D has come into contact with an object). Without staggered flexing of the fingers due to differences in drive radii, this could mean that other fingers (e.g. 3A, 3B or 3C) would not flex sufficiently to come into contact with the object being held (e.g. when the object is shaped like a cup or a glass). However, in the embodiment shown in the Figures, because of the staggered flexing of the fingers such that the fingers flex at different rates, this scenario is avoided.

Although not shown in the Figures, it will be appreciated that the prosthetic hand 1 could be provided with a thumb to be attached to the thumb mount 4. The thumb could be movable. Movement of the thumb could also be controlled using operating cable 42. For example, it could be that a drive wheel for driving movement of the thumb is connected via a corresponding drive cable through the thumb mount 4 to the drive bar assembly 6, for example, by way of set screws 56.

The components of the prosthetic hand may be manufactured using any suitable materials and techniques known in the art. For example, core load-bearing components may be made of metals such as aluminium, titanium, brass, bronze, aluminium bronze or steel (including anodized and/or nitrided steel). Non-load bearing components may be made of metal or other materials such as plastic. The components may be manufactured by any suitable methods, including casting, machining or additive manufacturing.

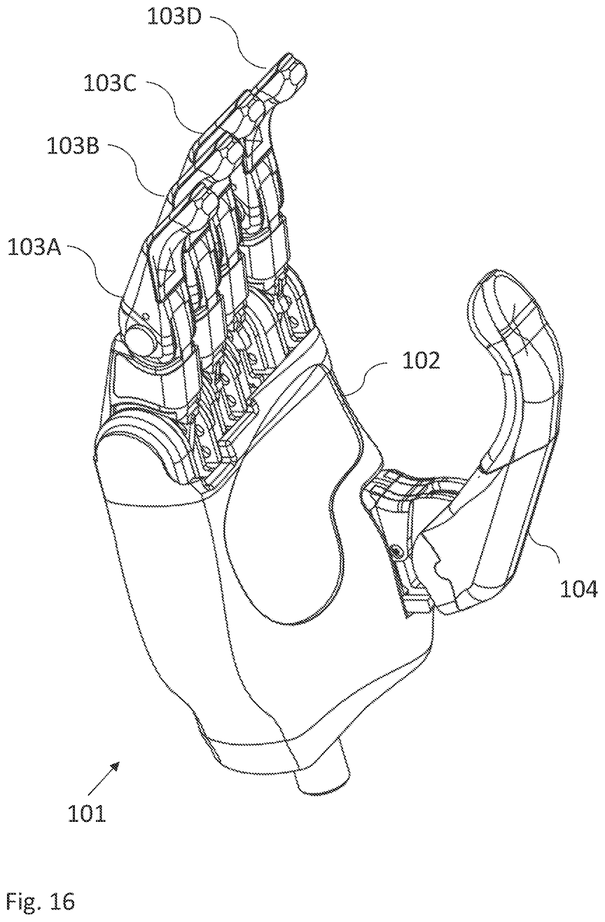

With reference to FIG. 16, a second example prosthetic hand 101 includes a hand body 102, four fingers 103A, 103B, 103C and 103D and a thumb 104. The prosthetic hand 101 is configured to be mounted onto a user's socket. Finger 103A corresponds to the pinkie or little finger of a human hand. Finger 103B corresponds to the ring finger of a human hand. Finger 103C corresponds to the middle finger of a human hand. Finger 103D corresponds to the index finger of a human hand.

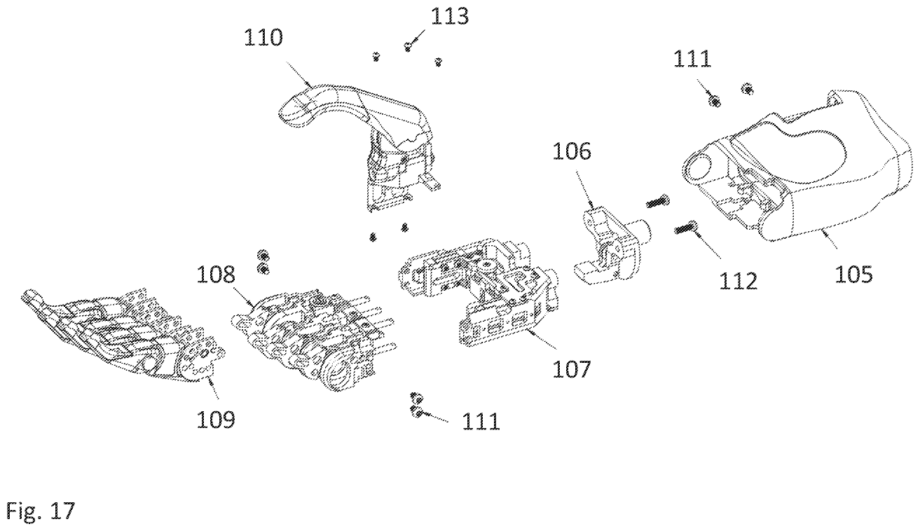

The prosthetic hand 101 is shown in more detail in FIGS. 17 to 26.

As shown in FIG. 17, the hand 101 includes a hand cover or housing 105, a wrist assembly 106 for mounting to a user's socket, a chassis or frame assembly 107, a knuckle assembly 108, a finger assembly 109 and a thumb assembly 110, which are fixedly mountable to one another by way of pan head screws 111 and 112 and button head screws 113.

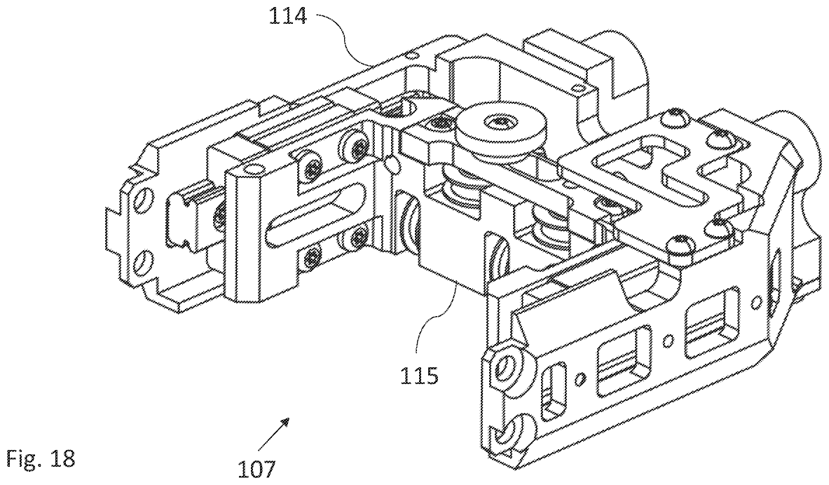

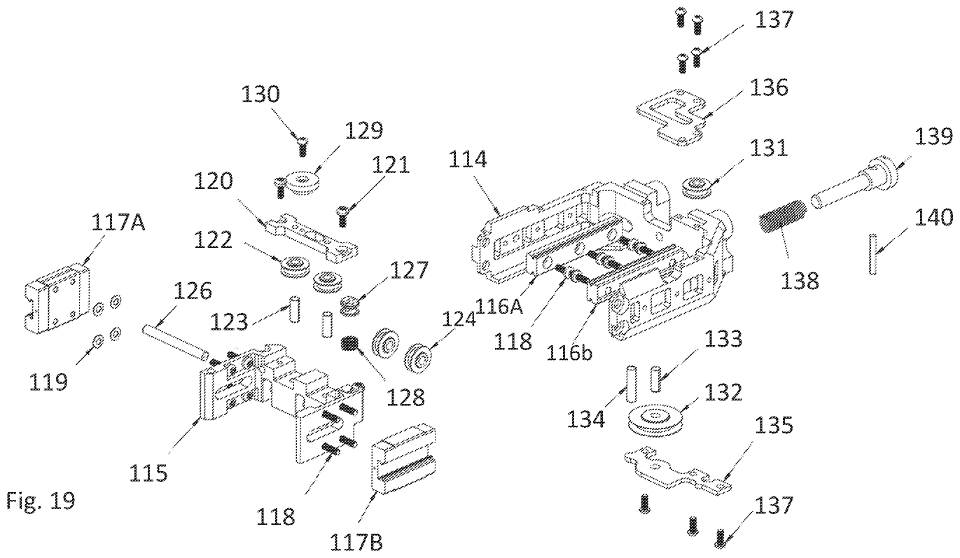

As shown in FIGS. 18 and 19, the chassis or frame assembly 107 includes a support frame or chassis 114 which supports a mechanism for controlling movement of the fingers 103A-103D.

The mechanism includes a U-shaped drive bar (a primary drive element) 115 which is slidably mountable within the U-shaped frame 114 on guide rails 116A and 116B. Guide rails 116A and 116B are fixed to the frame 114 by pan head screws 118. The drive bar 115 is slidably mountable on guide rails 116A and 116B by corresponding drive rail carriages 117A and 117B which are fixed to the drive bar 115 by pan head screws 118 together with spring washers 119.

A drive bar cap 120 is attached to the drive bar 115 by pan head screws 121. Drive bar cap 120 holds redirect pulleys 122 in place against the drive bar 115 on dowel pins 123. Redirect pulleys 124 are held within the drive bar 115 on dowel pin 126. Tension barrel 127 is also held within the drive bar 115 against redux spring 128. Thumb spigot 129 is attached to drive bar cap 120 by button head screw 130. A redirect pulley 131 and an amplifier pulley 132 are mounted within the frame 114 on dowel pins 133 and 134 between closure plate 135 and brace plate 136 attached to the frame 114 by buttonhead screws 137. A drive bar spring 138 is mounted on spring spur 139 through which dowel pin 140 is inserted.

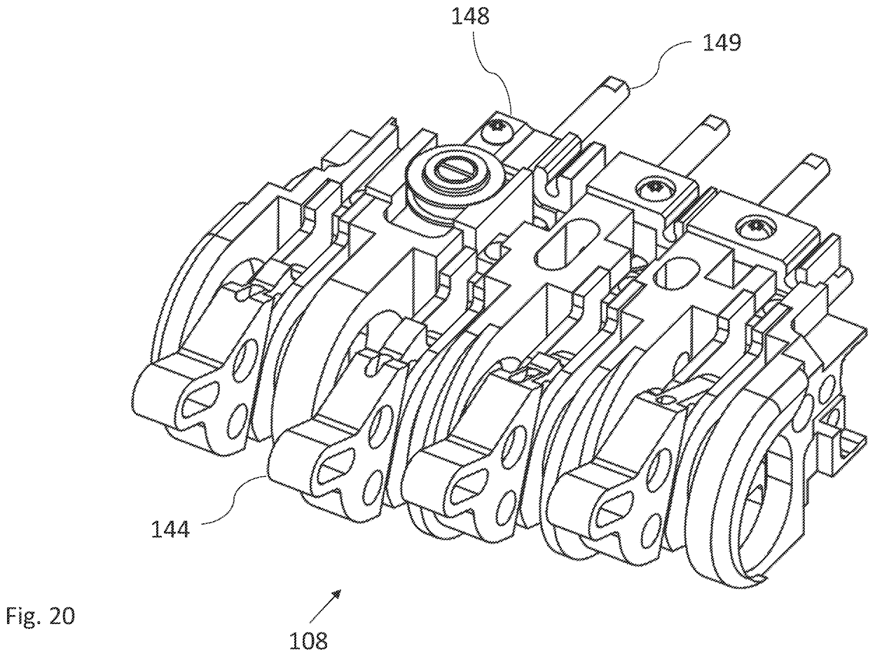

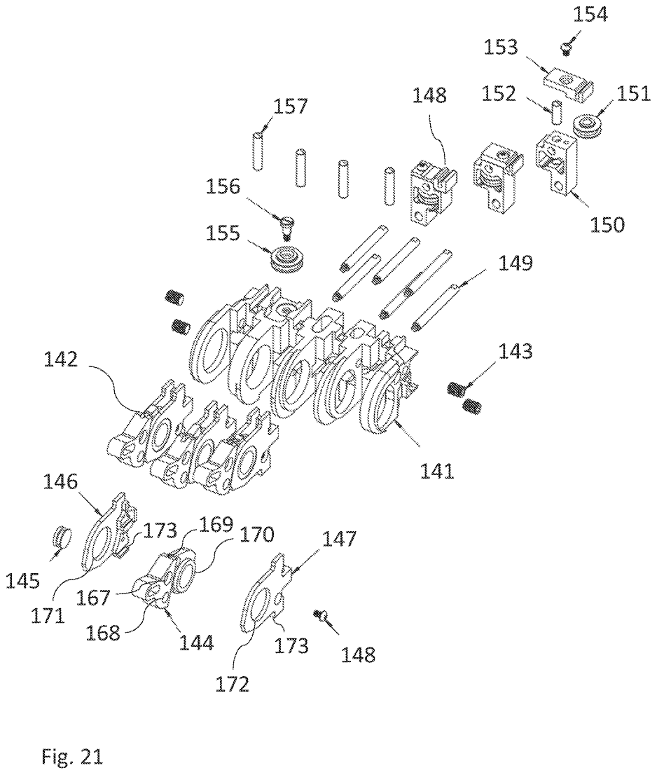

As shown in FIGS. 20 and 21, the knuckle assembly 108 includes a knuckle mount 141 to which individual knuckle bodies 142 are mountable by grub screws 143. Each knuckle body 142 includes a drive body 144 and cable bobbin 145 held between knuckle core plates 146 and 147 joined by button head screw 147.

The drive body 144 is shown in more detail in FIGS. 21, 26 and 27. Each drive body 144 includes a drive cable recess 167 for receiving the cable bobbin 145 and a return cable recess 168. A hollow channel 180 within the drive body 144 connects the drive cable recess 167 to a drive cable opening 169 on the surface of the drive body 144. A hollow channel 183 connects the return cable recess 168 to a return cable opening 184 on an opposing surface of the drive body 144. Each drive body 144 also includes a drive wheel portion 170 which is mountable and freely rotatable within corresponding apertures 171 and 172 in the knuckle core plates 146 and 147. The knuckle core plates 146 and 147 also include grooves return cable grooves 173 and 174 which, when the knuckle core plates 146 and 147 are joined, form a return cable slot.

Finger slider assemblies (secondary drive elements) 148 are slidably mounted on guide pins 149 fixedly attached at one end to the knuckle mount 141. Each finger slider assembly 148 includes a slider body 150 within which a redirect pulley wheel 151 is mounted on a dowel pin functioning as a pulley axle 152. The finger slider assembly 148 also includes a set screw 18 for adjusting the tension on a cable to be supported by the pulley 15. A finger dogleg 153 is attached to the slider body 150 by buttonhead screw 154. A thumb redirect pulley 155 is also attached to the knuckle mount 141 by shoulder screw 156. A corresponding dowel pin 157 for each finger slider assembly 148 is also inserted the knuckle mount 141.

In contrast to the first example prosthetic hand 1, in which each knuckle assembly 26 includes both a drive wheel 33 and a return wheel 34 between which a finger latch 31 is mounted, each knuckle body 142 of the second example prosthetic hand 101 includes a single drive body 144 to which a finger may be attached. The single drive body 144 is capable of driving movement of the finger in either direction, i.e. it functions as both a drive wheel and a return wheel.

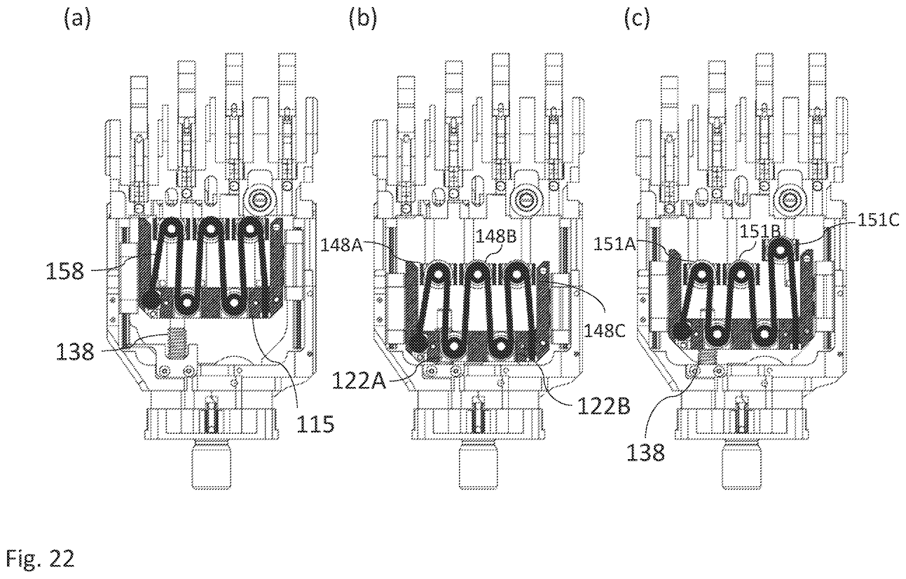

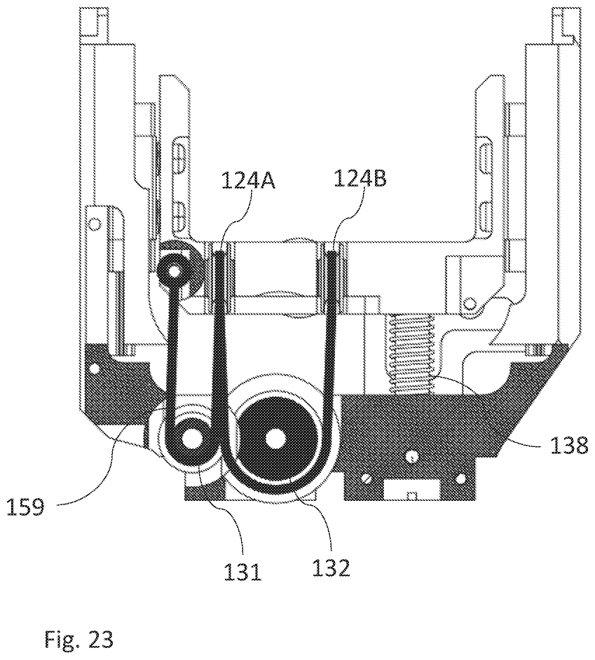

The prosthetic hand 101 is a cable-operated prosthetic hand. Thus, as illustrated in FIGS. 22 and 23, various components of the mechanism for controlling movement of the fingers are operably coupled to one another by way of cables. The prosthetic hand 101 operates in substantially the same way as prosthetic hand 1.

For example, as shown in FIG. 22, each of the finger slider assemblies 148A, 148B and 148C (corresponding to fingers 103A, 103B and 103C, respectively) is coupled to the drive bar 115 by a coupling cable 158 supported by the pulley wheels 151A, 151B, 151C, 122A and 122B and fixed at each end to the drive bar 115 (at one end via the tension barrel 127 for applying tension to the coupling cable 158). Each finger slider assembly 148 is also coupled to the corresponding knuckle body 142 by a corresponding drive cable 181 attached to the slider assembly via finger dogleg 153 and looped around the cable bobbin 145 within drive cable recess 167. The finger dogleg 153 ensures that the drive cable 181 is correctly aligned with the drive body 144 for the corresponding finger even though there may be an offset between the finger slider assemblies and the drive bodies. The drive bar 115 is also coupled directly to the knuckle body 142 corresponding to index finger 103D by a drive cable (not shown) attached directly to the drive bar 115. Each drive body 144 also includes a return cable 182 knotted at one end within return cable recess 168 and gripped at the opposite end within the return cable slot formed by return cable grooves 173 and 174. The return cable is formed from tensioned elastic or bungee cord.

When the prosthetic hand 101 is assembled, the hand is operable to provide control of movement of the fingers 103A to 103D via the corresponding knuckle assemblies 142 by applying tension to, or releasing tension from, operating cable 159, thus driving sliding movement of the drive bar assembly 115 along guide rails 116A and 116B. Movement of the drive bar assembly 115 can drive sliding movement of the finger slider assemblies 148A, 148B and 148C on the guide pins 149 when the finger slider assemblies are free to move. Movement of a finger slider assembly 148 in the driving direction D applies tension to the corresponding drive cable 181 which causes the corresponding drive body 144 to rotate between the knuckle core plates 146 and 147 (when the corresponding finger is free to move) in a first sense. Rotation of the drive body 144 in the first sense applies tension to the elastic return cable 182. When the tension applied to the drive cable 181 is released, the tension in the elastic return cable 182 urges rotation of the drive body 144 between the knuckle core plates 146 and 147 in the opposite sense. The knuckle assemblies 142 can therefore be considered as “self-returning” and the drive body 144 can be considered to function as both a drive wheel and a return wheel.

FIG. 22 (a) illustrates a configuration of the prosthetic hand 101 in which the drive bar assembly 115 has travelled as far as possible in return direction R. FIG. 22 (b) illustrates a configuration of the prosthetic hand 101 in which the drive bar assembly 6 has travelled as far as possible in the driving direction D, and in which all of the finger slider assemblies 148 have also travelled as far as possible in the driving direction D. FIG. 22 (c) illustrates a configuration of the prosthetic hand 101 in which the drive bar assembly 6 has travelled an intermediate distance in the driving direction D, and in which finger slider assembly 148C has not travelled as far as finger slider assemblies 148A and 148B (for example, because movement of the corresponding finger 103C has been blocked). The drive bar spring 138 is compressed as the drive bar assembly 115 moves in the driving direction D, biasing the drive bar assembly 115 towards the return direction R.

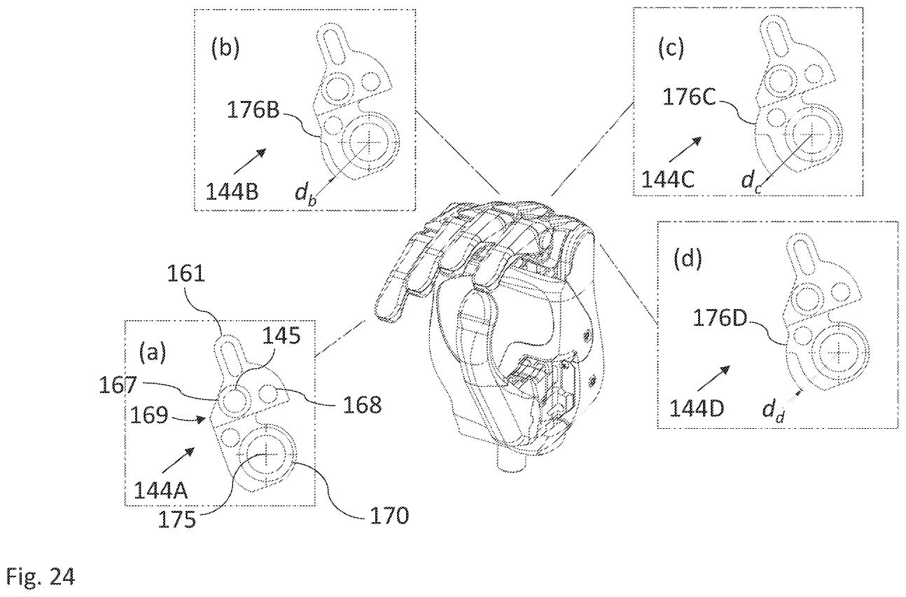

The mechanism for attaching and driving movement of the fingers is shown in more details in FIGS. 24 to 27.

As shown in FIG. 24, each drive body 144 has a drive wheel portion 170 and an attachment portion 161. The drive wheel portion 170 is the portion of the drive body 144 which is configured to be mounted within apertures 171 and 172 in the corresponding knuckle core plates 146 and 147 and defines the central axis 175 about which the drive body 144 rotates in use. The attachment portion 161 is configured for attaching a corresponding finger.

As described hereinabove, the drive cable 181 is attached to the drive body 144 by way of the cable bobbin 145 mounted within drive cable recess 167. The drive cable 181 extends from the cable bobbin 145 through the hollow channel within the drive body 144 and exits the drive body 144 at the drive cable opening 169.

As illustrated in FIG. 24, the drive bodies 144A, 144B, 144C and 114D of the knuckle bodies 142A, 142B, 142C and 142D (corresponding to fingers 103A, 103C, 103C and 103D) are not necessarily identical and can differ in terms of drive radii d (for example, drive body 144B has drive radius d_{b}, drive body 144C has drive radius d_{c}, and so on). The drive radius d of a given drive body is not simply the radius of the drive wheel portion 170 but is instead defined as the shortest straight line distance between the central axis 175 of the drive wheel portion 170 (about which the drive wheel portion rotates in the knuckle assembly) and the location where the drive cable 181 exits the drive cable opening 169.

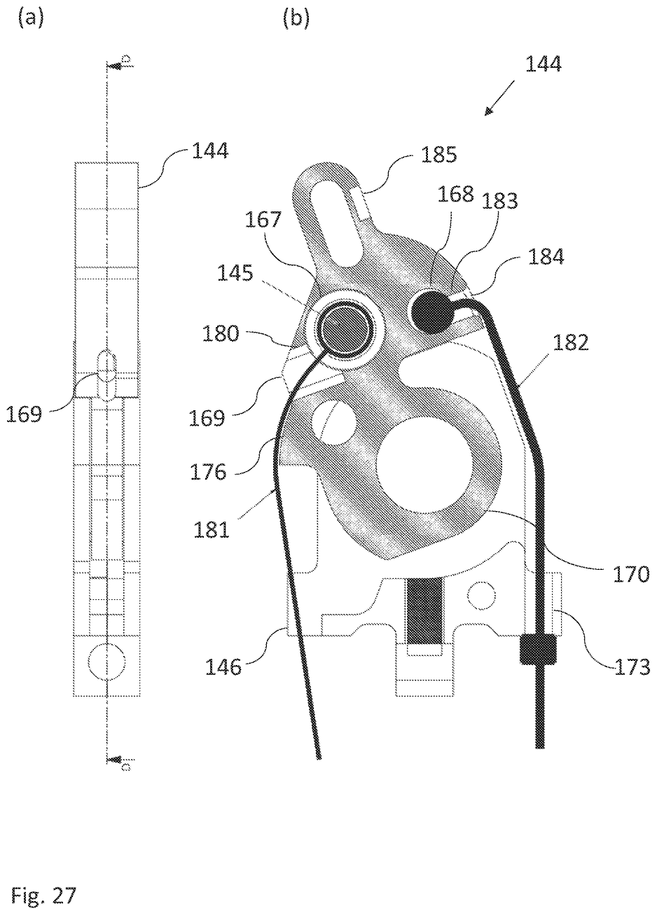

It will be appreciated that the location where the drive cable 181 exits the drive cable opening 169 may also define the location of a circumferential drive surface of the drive body against which the drive cable 181 may rest immediately after it exits the drive cable opening 169. However, in practice, this circumferential drive surface may not be physically present or only a portion of this circumferential drive surface may be physically present. For example, as shown in FIGS. 24 (b), (c) and (d) and in FIG. 27 (b), only a small portion 176B, 176C, 176D and 176, respectively, of the circumferential drive surface is physically present. The physical presence of a portion of the circumferential drive surface may be help ensure that the drive cable (and thus the applied load) remains tangential to the drive wheel portion 170 as the drive body 144 rotates. The remainder of the circumferential drive surface may be missing (i.e. the drive body 144 may be substantially non-circular in cross section) to reduce the amount of material used and thus the weight of the drive body 144.

It will be appreciated that, as the drive radius for a given drive wheel is reduced, the angular rotation of the drive wheel about its axis increases for a given drive cable displacement. Accordingly, when the drive radii of the drive wheels are different, all of the fingers are free to move, and tension is applied to the operating cable 159 to cause the drive bar assembly 115 and the finger slider assemblies 148A, 148B and 148C to move towards the base of the hand frame 114 at the same rate, the fingers will be rotated about their respective drive wheel axes at different (i.e. angular) rates.

The shape of the drive body 144 and/or the knuckle plates 146 and 147 may be configured to permit rotation of the drive body 144 through a particular range of angles. For example, the drive body 144 may be rotatable through up to about 70° of rotation to enable sufficient flexion and extension of the fingers.

Movement of the thumb assembly 110 may also be achieved by coupling the thumb 110 to the drive bar assembly 115 by a thumb cable (not shown) supported by the thumb redirect pulley 155.

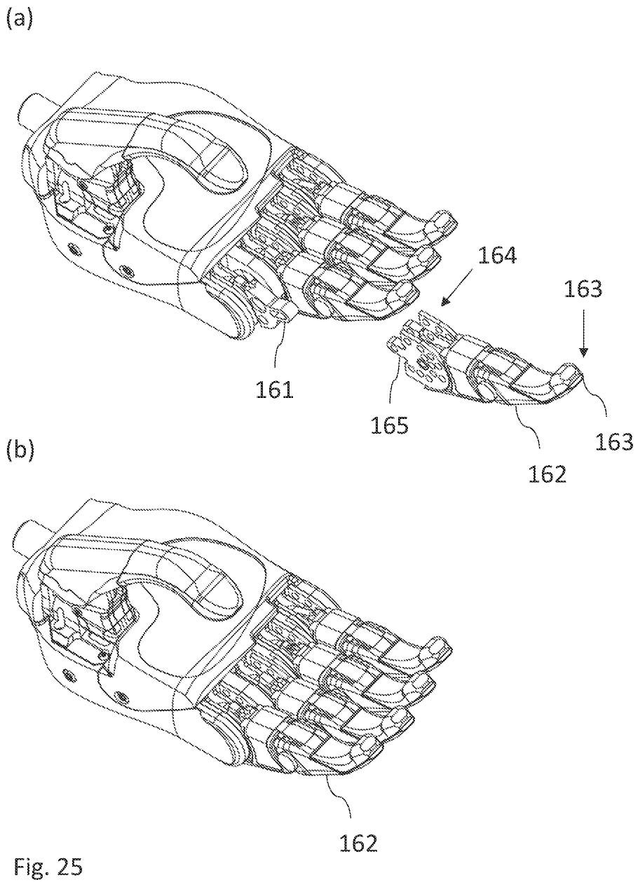

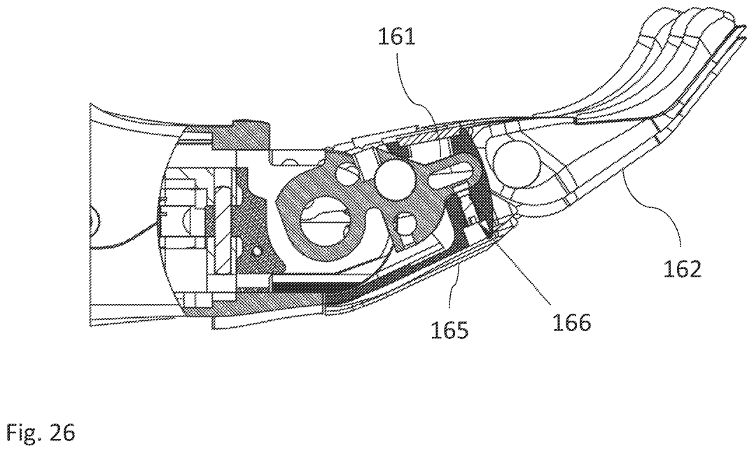

As shown in FIGS. 25 and 26, each finger includes a finger body 162 which extends between a distal end 163, at a fingertip, and a proximal end 164, at an attachment portion 165. The attachment portion 165 of the finger is configured for attachment to a corresponding attachment portion 161 of a drive body 144. In the example shown in FIGS. 25 and 26, the attachment portion 165 of the finger has a hollow core and the finger is attached by slotting the protruding attachment portion 161 of the drive body 144 into the hollow core and fixedly attaching the finger body 162 to the attachment portion 161 at 185 using grub screw 166. The finger can be detached by removing the grub screw 166 and sliding the finger off the hand. It will be appreciated that various other attachment and fastening methods are possible.

It will be appreciated that, once a finger is locked into place in the knuckle assembly, the finger is movable relative to the hand body 105 by applying or releasing tension to the corresponding drive cable and thus driving rotation of the corresponding driving wheel portion of the drive body (as discussed hereinabove). The direction of motion may depend on the shape of the finger and the design and placement of the knuckle assembly.

The components of the prosthetic hand may be manufactured using any suitable materials and techniques known in the art. For example, core load-bearing components may be made of metals such as aluminium, titanium, brass, bronze, aluminium bronze or steel (including anodized and/or nitrided steel). Non-load bearing components may be made of metal or other materials such as plastic. The components may be manufactured by any suitable methods, including casting, machining or additive manufacturing.

It will be understood that the invention is not limited to the embodiments described above and various modifications and improvements can be made without departing from the concepts described herein. Except where mutually exclusive, any of the features may be employed separately or in combination with any other features and the disclosure extends to and includes all combinations and sub-combinations of one or more features described herein.

For example, in some embodiments, it could be that none of the fingers are coupled directly to the drive bar assembly and instead all of the fingers could be coupled to the drive bar assembly by way of finger sliding assemblies. Alternatively, in other embodiments, it could be that more than one of the fingers is coupled directly to the drive bar assembly.

It could be that the drive wheels of the knuckle assemblies all have the same drive radii such that the fingers are configured to rotate about respective axes at the same rate. Alternatively, it could be that any of the knuckle assemblies have drive wheels having different drive radii. Moreover, it will be appreciated that the drive radius of a drive wheel could be varied in different ways by changing the shape and/or dimensions of the drive wheel.

It could be that one or more (e.g. all) of the fingers are permanently fixed to the hand body. Alternatively, it would be one or more (e.g. all) of the fingers are removably attached to the hand body by alternative mechanisms. It will be appreciated that any suitable connection mechanism which enables movement of the fingers could be used.

The prosthetic hand 1 or 101 could be a body-powered prosthetic hand (for example, where tension can be applied to or released from operating cable 42 or 159 by movements of a user's body when the hand is connected to a socket worn by the user) or an electrically powered prosthetic hand (for example, where an electric motor is used to apply tension to or release tension from the operating cable 42 or 159).

1. A prosthetic hand, comprising:
a hand body;
a plurality of fingers; and
a mechanism for actuating movement of the plurality of fingers relative to the hand body, wherein:
the mechanism comprises:
a movable primary drive element comprising one or more primary drive pulleys, and
a plurality of movable secondary drive elements each comprising a corresponding secondary drive pulley,
each of the plurality of secondary drive elements is coupled to the primary drive element by a coupling cable supported by the primary and secondary drive pulleys, such that movement of the primary drive element in a driving direction away from the secondary drive elements exerts a force on the secondary drive elements urging movement of the secondary drive elements in the driving direction,
each of the plurality of secondary drive elements is coupled to a corresponding finger of the plurality of fingers such that movement of a secondary drive element in the driving direction drives movement of the corresponding finger in a first direction relative to the hand body, and
the primary drive element and the secondary drive elements are mounted on one or more guides which constrain a direction of movement of the primary drive element and the secondary drive elements.

2. The prosthetic hand according to claim 1, wherein each finger of the plurality of fingers which is coupled to a secondary drive element is coupled to the corresponding secondary drive element by a corresponding coupling mechanism configured such that two or more of the plurality of fingers are moved in the first direction relative to the hand body at different rates from one another when the corresponding two or more secondary drive elements are moved in the driving direction at the same rate as one another.

3. The prosthetic hand according to claim 2, wherein:
the coupling mechanism for each finger comprises a corresponding drive wheel for driving movement of the said finger in the first direction,
each drive wheel is coupled to the corresponding secondary drive element by a corresponding drive cable, and
two or more of the drive wheels have different drive radii such that the corresponding two or more fingers are moved in the first direction relative to the hand body at different rates from one another when the corresponding two or more secondary drive elements are moved in the driving direction at the same rate as one another.

4. The prosthetic hand according to claim 3, wherein the drive wheel of an outermost finger of the plurality of fingers has the smallest drive radius.

5. The prosthetic hand according to claim 4, wherein the outermost finger corresponds to a little finger.

6. The prosthetic hand according to claim 1, wherein:
the plurality of fingers is a plurality of secondary fingers, and
the prosthetic hand further comprises a primary finger coupled directly to the primary drive element such that movement of the primary drive element in the driving direction drives movement of the primary finger in the first direction relative to the hand body.

7. The prosthetic hand according to claim 6, wherein the primary finger corresponds to an index finger.

8. The prosthetic hand according to claim 1, wherein the prosthetic hand comprises an operating cable coupled to the primary drive element and operable to move the primary drive element in the driving direction.

9. The prosthetic hand according to claim 8, wherein the operating cable is coupled to the primary drive element by one or more pulleys, levers, cams, screws, gear chains or joints, or any combination thereof.

10. The prosthetic hand according to claim 1, wherein the primary drive element and the plurality of movable secondary drive elements are biased in a return direction which opposes the driving direction.

11. The prosthetic hand according to claim 1, wherein the plurality of fingers is removably mounted on the hand body.

12. The prosthetic hand according to claim 1, further comprising a thumb and a thumb mechanism for actuating movement of the thumb.

13. The prosthetic hand according to claim 1, wherein the prosthetic hand is a body-powered prosthetic hand.

14. (canceled)

15. (canceled)

16. The prosthetic hand according to claim 1, wherein the prosthetic hand is an electrically powered prosthetic hand.

17. The prosthetic hand according to claim 16, further comprising a motor for driving movement of the primary drive element and/or an operating cable coupled to the primary drive element, and operable to move the primary drive element in the driving direction.

18. The prosthetic hand according to claim 1, wherein the plurality of fingers are each biased in a second direction relative to the hand body which opposes the first direction.

19. A method of operating the prosthetic hand according to claim 1, the method comprising applying a driving force to the primary drive element to cause movement of the primary drive element in the driving direction, wherein movement of the primary drive element in the driving direction drives movement of one or more of the secondary drive elements in the driving direction via the coupling cable, and wherein movement of the one or more of the secondary drive elements in the driving direction drives movement of the one or more corresponding fingers in the first direction relative to the hand body.

20. The method according to claim 19, wherein the primary drive element and the plurality of movable secondary drive elements are biased in a return direction which opposes the driving direction.

21. The method according to claim 20, wherein the plurality of fingers are each biased in a second direction relative to the hand body which opposes the first direction, and wherein the method further comprises reducing or releasing the driving force applied to the primary drive element to permit movement of the primary drive element and the one or more of the secondary drive elements in the return direction and movement of the one or more corresponding fingers in the second direction relative to the hand body.
